SEQUENCE LISTING (2 sequences)

SEQ ID NO: 1 - protein, 20 aa, Artificial Sequence
  Artificial streptavidin sequence
IDARRASVGTSAWRHPQFGG

SEQ ID NO: 2 - protein, 5 aa, Artificial Sequence
  Artificial poly-histidine tag
HHHHH

(19) United States
(12) Patent Application Publication
(10) Pub. No.: US 2003/0216337 A1
(43) Pub. Date: Nov. 20, 2003

(54) Composition and imaging methods for pharmacokinetic and pharmacodynamic evaluation of therapeutic delivery system

(75) Inventor: Dennis E. Hallahan, Nashville, TN (US)

Correspondence Address: JENKINS & WILSON, PA, 3100 TOWER BLVD, SUITE 1400, DURHAM, NC, 27707, US

(73) Assignee: Vanderbilt University

(21) Appl. No.: 10/342,805

(22) Filed: Jan. 15, 2003

Related U.S. Application Data
(60) Provisional application No. 60/348,945, filed on Jan. 15, 2002.

(51) Int. Cl. A61K 51/00; A61K 48/00

(52) U.S. Cl. 514/44; 424/1.73

(57) ABSTRACT

A halogen-labeled gene therapy construct that includes halogen-labeled nucleic acids, methods for preparing a halogenated gene therapy construct, and methods for in vivo imaging of the same. Also provided are methods for non-invasive drug detection in a subject using a labeled antibody that recognizes a heterologous antigen conjugated to, encoded by, or otherwise associated with the drug.

### CROSS REFERENCE TO RELATED APPLICATIONS

This application is based on and claims priority to U.S. Provisional Application Serial No. 60/348,945, filed Jan. 15, 2002, herein incorporated by reference in its entirety.

### GRANT STATEMENT

This work was supported by grants R01-CA88076-01A2, R01-CA70937, R01-CA89674-01, R21-CA89888-01, 2-R01-CA58508, 2-P30-CA68485-04, and P50-CA90949 from the U.S. National Institutes of Health. Thus, the U.S. government has certain rights in the invention.

### FIELD OF THE INVENTION

The present invention generally relates to in vivo imaging of drug biodistribution. More particularly, the present invention relates to methods for drug labeling such that drug can be detected non-invasively following administration to a subject.

Table of Abbreviations

| | | |
|---|---|---|
| 3-D | - | 3-dimensional |
| Ad | - | adenovirus |
| ANOVA | - | Analysis of Variance |
| AR | - | autoradiography |
| CEA | - | carcinoembryonic antigen |
| CHCA | - | alpha-cyano-4-hydroxycinnamic acid |
| CPM | - | counts per minute |
| CT | - | computerized tomography |
| DHBA | - | 2,5-dihydroxybenzoic acid |
| DTPA | - | diethylenetriamine pentaacetate |
| EDC | - | 1-ethyl-3-(3-dimethylaminopropyl)- |
| | | carbodiimide |
| ELISA | - | enzyme-linked immunosorbent assay |
| ExFlk | - | soluble Flk-1 receptor |
| ExFlk.6His | - | soluble Flk-1 receptor |
| ExTek | - | soluble portion of TEK/Tie2 receptor |
| ExTek.Strep | - | soluble portion of TEK/Tie2 receptor fused |
| | | to Streptavidin antigenic peptide |
| GEE | - | Generalized Estimating Equation |
| GM-CSF | - | granulocyte-macrophage colony- |
| | | stimulating factor |
| HLA | - | human leukocyte antigen |
| HPLC | - | high performance liquid chromatography |
| IL-2 | - | interleukin 2 |
| IL-4 | - | interleukin 4 |
| IL-7 | - | interleukin 7 |
| IL-12 | - | interleukin 12 |
| IPs | - | imaging plates |
| IRES | - | internal ribosome entry site |
| ITR | - | inverted terminal repeat |
| IUdR | | iodinated uridine deoxyribonucleic acid |
| keV | - | kilo electron volts |
| kVp | - | kilovolt peak |
| LTR | - | long terminal repeat |
| μCi | - | microcurie |
| mA | - | milliamp(s) |
| MALDI | - | matrix-assisted laser desorption ionization |
| | | mass spectrometry |
| MALDI-TOF | - | matrix-assisted laser desorption ionization/ |
| | | time-of-flight mass spectrometry |
| mCi | - | millicurie |
| mEq | - | milliequivalent |
| m/z | - | mass to charge ratio |
| mg/m² | - | milligrams per square meter |
| MSIT | - | Mass Spectrometry Image Tool |
| NM | - | nuclear magnetic |
| ODs | - | optical densities |
| PBS | - | phosphate buffered saline |
| PD | - | pharmacodynamic(s) |
| PEG | - | polyethylene glycol |
| PET | - | positron emission spectroscopy |
| PFU | - | plaque-forming units |
| PK | - | pharmacokinetic(s) |
| REML | - | restricted/residual maximum likelihood |
| SAS | - | Statistical Analysis System |
| SD | - | standard deviation |
| Sn-UdR | - | 5-Tributylstannyl-2-deoxyuridine |
| SPDP | - | succinimidyl 3-(2-pyridyldithio)propionate |
| SPECT | - | single photon emission computed |
| | | tomography |
| TEK/Tie2 | - | angiopoietin-1 receptor |
| TNF | - | tumor neorosis factor |
| TNF-α | - | tumor necrosis factor alpha |
| VEGF | - | vascular endothelial growth factor |

### BACKGROUND OF THE INVENTION

A limitation of current therapeutic methods is that drug biodistribution following administration to a subject can be non-specific or non-homogenous. For example, local injection of a gene therapy construct can result in a non-homogeneous distribution of an encoded gene product along the injection track. Systemic administration of a gene therapy construct can improve the distribution of an encoded gene product, although the construct dose achieved at a target tissue is unpredictable. In addition, systemic toxicity can result from vector delivery to non-target tissues.

To facilitate effective provision of therapeutic agents, a method for in vivo monitoring of drug biodistribution has been sought. Thus, there exists a long-felt need in the art for methods for drug labeling that are suitable for non-invasive imaging following administration to a subject.

To meet this need, the present invention provides a halogen-labeled gene therapy construct and methods for preparing and for in vivo imaging of the same. Also provided are methods for non-invasive drug detection in a subject using a labeled antibody that recognizes a heterologous antigen conjugated to, encoded by, or otherwise associated with the drug.

### SUMMARY OF THE INVENTION

The present invention provides a method for preparing a halogen-labeled gene therapy construct. The method comprises: (a) introducing a gene therapy construct into helper cells, wherein the gene therapy construct comprises one or more nucleic acids; and (b) providing a halogen-labeled nucleotide to the helper cells, whereby a halogen-labeled gene therapy construct is prepared. The method can further comprise isolating the halogen-labeled gene therapy construct from the helper cells.

Also provided are halogen-labeled gene therapy constructs produced by the disclosed labeling method. Such halogen-labled gene therapy constructs comprise: (a) a vector; and (b) one or more nucleic acids, wherein the nucleic acids comprise a halogen-labeled nucleotide, wherein the nucleic acids are free of triplex structures, and wherein the halogen-labeled gene therapy construct can be detected in vivo.

Also provided is a method for non-invasive detection of a halogen-labeled gene therapy construct following administration to a subject. The method includes the steps of: (a) administering to a subject an effective dose of a halogen-labeled gene therapy construct, wherein the gene therapy construct comprises a vector and one or more nucleic acids, and wherein one or more of the nucleic acids comprises a halogen-labeled nucleotide; and (b) detecting the halogen-labeled nucleotide, wherein the detecting comprises a non-invasive detection technique, whereby the gene therapy construct in a subject is detected non-invasively.

In accordance with the disclosed compositions and methods, a halogen-labeled gene therapy construct can comprise a viral vector, a plasmid, a liposome, or combinations thereof. In one embodiment of the invention, the vector comprises an adenoviral vector.

The nucleic acids of the gene therapy construct can comprise a nucleotide sequence encoding a therapeutic gene product, for example a therapeutic polypeptide or a therapeutic oligonucleotide.

A gene therapy construct of the present invention comprises nucleic acids comprising a halogen-labeled nucleotide. The halogen can comprise a radiohalogen. In one embodiment, a radiohalogen comprises ¹⁸fluorine, in another embodiment ¹²³iodine, in another embodiment ¹²⁵iodine, and in still another embodiment ¹³¹iodine. In one embodiment of the invention, the halogen-labeled nucleotide comprises a pyrimidine nucleoside. In another embodiment, the halogen-labeled nucleotide comprises 2′-deoxyuridine.

The present invention further provides a method for drug detection in a subject using non-invasive imaging methods, the method comprising: (a) administering to a subject an effective dose of a drug, wherein the drug comprises a heterologous antigen; (b) administering to the subject an antibody that binds the heterologous antigen, wherein the antibody comprises a label that can be detected in vivo; and (c) detecting the label in vivo, whereby the drug is detected in the subject.

The drug can comprise a nucleic acid (e.g., a gene therapy construct and/or a nucleic acid comprising a nucleotide sequence encoding a therapeutic gene product), a small molecule, a protein, a peptide, a lipid, or combinations thereof.

The heterologous antigen comprises any antigen not normally present in the subject. In one embodiment of the invention, the heterologous antigen comprises a streptavidin peptide. In one embodiment, a streptavidin peptide comprises an amino acid sequence of SEQ ID NO: 1. In another embodiment of the invention, the heterologous antigen comprises a polyhistidine peptide. In one embodiment, a polyhistidine peptide comprises an amino acid sequence of SEQ ID NO: 2.

In one embodiment, the antibody used to perform the method specifically binds a heterologous antigen comprising a streptavidin peptide, such as a peptide comprising an amino acid sequence of SEQ ID NO: 1 or 2. Antibodies include, but are not limited to Fab fragments and single chain antibodies. According to the method, the antibody further comprises a label that can be detected in vivo, for example by using any one of techniques including but not limited to magnetic resonance imaging, scintigraphic imaging, ultrasound, fluorescence, and combinations thereof.

When scintigraphic imaging is employed, the detectable label comprises in one embodiment a radionuclide label. In one embodiment, a radiohalogen comprises ¹⁸fluorine, in another embodiment ¹²³iodine, in another embodiment ¹²⁵iodine, and in still another embodiment ¹³¹iodine. According to the disclosed methods, the radionuclide label can be detected using positron emission tomography, single photon emission computed tomography, gamma camera imaging, rectilinear scanning, or combinations thereof.

In accordance with the disclosed methods for non-invasive imaging of drug distribution, including a distribution of a halogen-labeled gene therapy construct, an effective dose comprises a detectable amount of the drug, wherein the detectable amount is determined using non-invasive imaging.

The disclosed methods are suitable for detection of a therapeutic and/or diagnostic composition following administration to a warm-blooded vertebrate subject, in one embodiment a human subject.

Accordingly, it is an object of the present invention to provide halogen-labeled gene therapy constructs, antigen-labeled gene therapy constructs and other drugs, and methods for in vivo imaging of drug distribution following administration to a subject. This object is achieved in whole or in part by the present invention.

An object of the invention having been stated above, other objects and advantages of the present invention will become apparent to those skilled in the art after a study of the following description of the invention, Figures, and non-limiting Examples.

### BRIEF DESCRIPTION OF THE DRAWINGS

FIGS. 1A-1D are images of rat subjects following intratumoral administration of radiohalogenated adenoviral vector. Tumors were implanted into hind limbs, and ¹³¹I-labeled Ad.ExFlk was injected intratumorally and/or intravenously. Images were obtained as described in Example 2. CT images are viewed in grayscale, and overlaid SPECT images are viewed in color (here, arrows).

FIG. 1A is a CT/SPECT image of a top view of two rat subjects in the imaging chamber. The subjects are positioned nose-to-nose, such that the top of the panel corresponds to the posterior of the first animal, the bottom of the panel corresponds to the posterior of the second animal, and the middle of the panel corresponds to the adjacent noses of the first and second animals. Radiohalogenated Ad.ExFlk.6His was administered intratumorally to the first animal (top). Radiohalogenated Ad.ExFlk.6His was administered by tail vein injection to the second animal (bottom). Ad.ExFlk.6His was detected in the tumor of the first animal (top, thin arrow) and in the liver of the second animal (bottom, thick arrow).

FIG. 1B is a CT image of a coronal section of the second animal in FIG. 1A, the cross-section being taken through the spleen. The spinal cord appears as a bright spot (asterisk), and the spleen is positioned ventral to the spleen (arrow).

FIG. 1C is a SPECT image of the same coronal section pictured in 1B. Radiohalogenated Ad.ExFlk.6His appears as regions of gray signal. When viewed in color, radiohalogenated Ad.ExFlk.6His appears as regions of red and orange hues (arrow). In this view, radiohalogenated Ad.ExFlk.6His is detected in the spleen (arrow).

FIG. 1D is a SPECT image of a coronal second of the first animal in FIG. 1A, the section being taken through the tumor in the hind limb. Radiohalogenated Ad.ExFlk.6His appears as regions of gray signal. When viewed in color, radiohalogenated Ad.ExFlk.6His appears as regions of red and orange hues (arrow). In this view, radiohalogenated Ad.ExFlk.6His is detected in the tumor (arrow).

FIGS. 2A-2B are images of sections of a C6 glioma in rat hind limb. Radiohalogenated Ad.ExFlk.6His was prepared as described in Example 1 as was administered intratumorally to a rat subject.

FIG. 2A is a MALDI mass spectrophotometric image of the C6 glioma obtained as described in Example 4. Localization of the radiolabeled antibody in the tumor is observed as regions of white or gray signal.

FIG. 2B is an optical image of the same C6 glioma depicted in FIG. 2A. The optical image enables visualization of the tumor (area outlined by arrowheads).

FIG. 3 is an immunoblot depicting ExFlk.6His protein immunoprecipitated using a ANTI-PENTA-HIS™ antibody (Qiagen Inc., Valencia, Calif., United States of America). Adenovirus encoding ExFlk.6His was administered to a rat subject by tail vein injection. Total protein was extracted from tissues, and proteins were immunoprecipitated using a monoclonal PENTA-HIS™ antibody (Qiagen, Inc., Valencia, Calif., United States of America) as described in Example 5. Immunoprecipitated ExFlk and IgG were resolved by denaturing gel electrophoresis. Lanes 1-8 depict immunoprecipitates from: (1) recombinantly produced ExFlk.6His (positive control); (2) liver; (3) kidney; (4) lung; (5) muscle; (6) heart; (7) spleen; (8) whole animal not receiving Ad.ExFlk.6His injection (negative control).

### BRIEF DESCRIPTION OF THE SEQUENCE LISTING

SEQ ID NO: 1 is the amino acid sequence of an artificial streptavidin peptide.

SEQ ID NO: 2 is an amino acid sequence of a poly-histidine tag.

### DETAILED DESCRIPTION OF THE INVENTION

I. Definitions

While the following terms are believed to be well understood by one of ordinary skill in the art, the following definitions are set forth to facilitate explanation of the invention.

The terms “nucleic acid material” and “nucleic acids” each refer to deoxyribonucleotides, ribonucleotides, or analogues thereof in either single- or double-stranded form. Unless specifically limited, the term encompasses nucleic acids containing known analogues of natural nucleotides that have similar properties as the reference natural or antisense nucleic acid. Thus “nucleic acids” includes but is not limited to DNA, cDNA, RNA, antisense RNA, and double-stranded RNA. A therapeutic nucleic acid can comprise a nucleotide sequence encoding a therapeutic gene product, including a polypeptide or an oligonucleotide.

Nucleic acids can further comprise a gene (e.g., a therapeutic gene), a drug delivery vehicle such as a gene therapy vector, or any other sequence that can be used as a diagnostic element. The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “expression”, as used herein to describe a gene therapy construct, generally refers to the cellular processes by which a biologically active polypeptide or biologically active oligonucleotide is produced from a DNA sequence.

The term “small molecule” as used herein refers to a compound, for example an organic compound, with a molecular weight of in one embodiment less than about 1,000 daltons, in another embodiment less than about 750 daltons, in another embodiment less than about 600 daltons, and in still another embodiment less than about 500 daltons. A small molecule also has a computed log octanol-water partition coefficient in one embodiment in the range of about −4 to about +14, in another embodiment in the range of about −2 to about +7.5, and is both water-soluble and lipid-soluble. In one embodiment, a small molecule comprises five or fewer hydrogen-bond donor sites, and fewer than ten atoms comprising nitrogen or oxygen.

The term “binding” refers to an affinity between two molecules, for example, between an antibody and an antigen. As used herein, “binding” means a preferential binding of one molecule for another in a mixture of molecules. The binding of a ligand to a target molecule can be considered specific if the binding affinity is about 1×10⁴ M⁻¹ to about 1×10⁶ M⁻¹ or greater.

The phrase “specifically (or selectively) binds”, for example when referring to the binding capacity of an antibody, refers to a binding reaction which is determinative of the presence of the antigen in a heterogeneous population of proteins and other biological materials. The phrase “specifically binds” also refers to selective targeting of a targeted molecule.

The phases “substantially lack binding” or “substantially no binding”, as used herein to describe binding of an antibody to a heterologous antigen, refers to a level of binding that encompasses non-specific or background binding, but does not include specific binding.

The term “subject” as used herein refers to any invertebrate or vertebrate species. The methods of the present invention are particularly useful in the treatment and diagnosis of warm-blooded vertebrates. Thus, the invention concerns mammals and birds. More particularly, contemplated is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, contemplated is the treatment of livestock, including, but not limited to domesticated swine (pigs and hogs), ruminants, horses, poultry, and the like.

The term “about”, as used herein when referring to a measurable value such as an amount of weight, time, dose, etc. is meant to encompass variations of in one embodiment ±20% or ±10%, in another embodiment ±5%, in another embodiment ±1%, and in still another embodiment ±0.1% from the specified amount, as such variations are appropriate to perform the disclosed methods.

II. Halogen-Labeled Gene Therapy Constructs

The present invention provides halogen-labeled gene therapy constructs and methods for producing the same. In one embodiment of the invention, the halogen comprises a radiohalogen that can be detected using scintigraphic imaging.

Current strategies for radiographic imaging of a gene therapy vector include: (a) transcription-based metabolism of a radionuclide substrate (U.S. Pat. No. 5,703,056); (b) encapsulation of a radionuclide-labeled bacterial peptidoglycans in liposomes (U.S. Pat. No. 5,017,359); (c) radionuclide labeling of a vector protein (Schellingerhout et al., 1998; Lerondel et al., 2001); and (d) hybridization-based labeling of vector nucleic acids to form triplex structures (PCT International Publication No. WO 99/61071).

Transcription-based methods have been useful to detect expression of a polypeptide encoded by a gene therapy vector, but do not identify cells or tissues that are successfully transformed but fail to express the reporter gene. Thus, such methods do not assay the physical biodistribution of the vectors, and an amount of virus injected and an amount of reporter gene expression can be poorly correlated. See e.g., MacLaren et al., 1999. Attachment or encapsulation of a detectable moiety has enabled direct assessment of the biodistribution of a gene therapy construct, even in cells that do not express the encoded gene. However, several technical limitations of vector labeling are apparent, including, for example, difficulty in attaching labeling moieties to some vectors (e.g., covalently closed, circular plasmids).

To obviate these shortcomings, the present invention provides a method for preparing a halogen-labeled gene therapy construct. The method comprises: (a) introducing a gene therapy construct into helper cells, wherein the gene therapy construct comprises one or more nucleic acids; and (b) providing a halogen-labeled nucleotide to the helper cells; whereby a halogen-labeled gene therapy construct is prepared. The labeling method is simple to perform and can readily be used for labeling any gene therapy construct comprising nucleic acids. The method can further comprise isolating the halogen-labeled gene therapy construct from the helper or host cells.

Also provided are halogen labeled gene therapy constructs produced by the disclosed labeling method. In one embodiment, a halogen-labeled gene therapy construct of the present invention comprises: (a) a vector; and (b) nucleic acids, wherein the nucleic acids comprise a halogen-labeled nucleotide, wherein the nucleic acids are free of triplex structures, and wherein the gene therapy construct can be detected in vivo.

The term “construct”, as used herein to describe a gene therapy construct, refers to a composition comprising a vector used for gene therapy. In one embodiment, the composition also includes nucleic acids comprising a nucleotide sequence encoding a therapeutic gene product, for example a therapeutic polypeptide or a therapeutic oligonucleotide. In one embodiment, the nucleotide sequence is operatively inserted with the vector, such that the nucleotide sequence encoding the therapeutic gene product is expressed. The term “construct” also encompasses a gene therapy vector in the absence of a nucleotide sequence encoding a therapeutic polypeptide or a therapeutic oligonucleotide, referred to herein as an “empty construct.” The term “construct” further encompasses any nucleic acid that is intended for in vivo studies, such as nucleic acids used for triplex and antisense pharmacokinetic studies.

II.A. Labeling Methods

In contrast to known vectors for in vivo imaging, the present invention provides a halogen-labeled gene therapy construct comprising nucleic acids, wherein the nucleic acids comprise a halogen-labeled nucleotide. Preparation of nucleic acids comprising a halogen-labeled nucleotide can be accomplished by any suitable method known in the art including but not limited to: (a) PCR amplification or nucleic acids in the presence of halogen-labeled nucleotides; (b) terminal transferase addition of halogen-labeled nucleotides to a nucleic acid; (c) recombinant production of halogen-labeled nucleic acids, for example, recombinant production in prokaryotic cell, eukaryotic cell, or plant cell systems; and (d) incorporation of halogen-labeled nucleotides during preparation of a viral vector. Halogen-labeled gene therapy constructs of the present invention are in one embodiment free of triplex structures, such as described in PCT International Publication No. WO 99/61071.

In one embodiment, a halogen-labeled nucleotide includes, but is not limited to a pyrimidine nucleotide, for example 2′-deoxyuridine. Thus, a representative nucleotide that can be used in accordance with the labeling methods disclosed herein is 5-Iodo-2′-deoxyuridine (IUdR), a thymidine analog in which the 5-methyl group of thymidine is replaced by iodine. IUdR specifically incorporates into DNA during the synthetic phase of the cell cycle. IUdR that has been incorporated into cellular DNA is retained for the life of the cell or its progeny. In contrast, unincorporated IUdR is rapidly catabolized to iodouracil and/or dehalogenated, the resulting compound having a short half-life (less than minutes in humans). The preparation of IUdR as well as iodinated versions is disclosed in U.S. Pat. No. 4,851,520.

In another embodiment, the halogen comprises a stable halogen, including F, Cl, Br, and I. In one embodiment of the invention, the halogen comprises a radiohalogen. The term “radiohalogen” refers to a radioactive isotope of a halogen or halide salt. The term “radioactive” refers to a quality of an atom in emitting photon α-particles, β-particles, or positrons. Thus, the term “radiohalogen” refers to radioactive isotopes of F, Cl, Br, and I, such as ¹⁸fluorine, ^{80m}bromine, ¹²³iodine, ¹²⁴iodine, ¹²⁵iodine, ¹²⁶iodine, ¹³¹iodine, ¹³³iodine, ⁷⁷iodine, and ^{80m}iodine. In one embodiment of the invention, a radiohalogen comprises ¹⁸fluorine, ¹²³iodine, ¹²⁵iodine, or 131iodine. Radiohalogens can be prepared using standard laboratory methods known to one of skill in the art.

The present invention also provides a method for preparing a halogen-labeled gene therapy construct, the method comprising: (a) introducing a gene therapy construct into helper cells, wherein the gene therapy construct comprises one or more nucleic acids; and (b) providing a halogen-labeled nucleotide to the helper cells; whereby a halogen-labeled gene therapy construct is prepared.

The term “helper cell” as used herein refers to a cell that is transduced with a gene therapy construct or a vector, wherein the helper cell can amplify the gene therapy construct or vector. Thus, the term “helper cell” includes prokaryotic, eukaryotic, and plant heterologous expression systems. The term “helper cell” also encompasses packaging cells used to prepare viral vectors, as described further herein below.

In one embodiment of the invention, a gene therapy construct comprises a viral vector. In one embodiment, a viral vector of the invention is disabled, e.g. helper-dependent. The term “helper-dependent” refers to a recombinant viral vector that is incapable of propagation in the absence of a helper functions. Thus, a helper-dependent viral vector typically comprises a deleted and/or altered genome, wherein one or more gene functions required for viral propagation are disrupted. For example, a representative helper-dependent adenoviral vector can comprise functional deletions in one or more of the adenovirus genes E2a, E4, the late genes L1 through L5, and/or the intermediate genes IX and IVa.

The terms “packaging cell” or “packaging cell line” refer to a cell line that permits or facilitates virus replication and packaging. A packaging cell line typically comprises trans-complementing functions that have been deleted from a helper-dependent virus. Suitable packaging lines for retroviruses include derivatives of PA317 cells, ψ-2 cells, CRE cells, CRIP cells, E-86-GP cells, and 293GP cells. Line 293 cells can be used for adenoviruses and adeno-associated viruses.

Nucleic acids, for example a nucleic acid encoding a therapeutic gene product, can be incorporated into viral genomes by any suitable means known in the art. Typically, such incorporation will be performed by ligating the construct into an appropriate restriction site in the genome of the virus. Viral genomes can then be packaged into viral coats or capsids by any suitable procedure.

Thus, a halogen-labeled adenoviral construct for gene therapy can be prepared by: (a) introducing a helper-dependent gene therapy construct into helper packaging cells, (b) providing a halogen-labeled nucleotide to the helper packaging cells, whereby a halogen-labeled gene therapy construct is prepared. Representative methods for preparing a halogen-labeled adenoviral gene therapy construct are described in Example 1. Briefly, packaging cells are infected with a viral vector, and ¹³¹IUdR is provided to the packaging cells, whereby a ¹³¹I-labeled adenovirus is produced. Following administration of a radiohalogenated gene therapy vector to a subject, the biodistribution of such a vector can be detected using scintigraphic methods, as described herein below under the heading Scintigraphic Imaging.

II.B. Therapeutic Nucleic Acids

In one embodiment of the invention, a halogen-labeled gene therapy construct further comprises a nucleotide sequence encoding a therapeutic polypeptide or a therapeutic oligonucleotide. Halogen-labeled gene therapy constructs can be used for the treatment of any condition wherein expression of a gene product having therapeutic or prophylactic activity is sought. Such constructs are particularly suited for treatment of tumors or other neoplasms.

Representative therapeutic oligonucleotides include, but are not limited to antisense RNA (Ehsan & Mann, 2000; Phillips et al., 2000), double-stranded oligodeoxynucleotides (Morishita et al., 2000), ribozymes (Shippy et al., 1999; de Feyter & Li, 2000; Norris et al., 2000; Rigden et al., 2000; Rossi, 2000; Smith & Walsh, 2000; Lewin & Hauswirth, 2001), and peptide nucleic acids (Ehsan & Mann, 2000; Phillips et al., 2000). Methods for the design, preparation, and testing of therapeutic oligonucleotides can be found in the sources listed herein above, and references cited therein, among other places.

Representative therapeutic polypeptides include those polypeptides that are abnormally absent or expressed at insufficient levels in a subject. A therapeutic polypeptide can also comprise a polypeptide that is antagonistic to an abnormal activity in a subject, for example unregulated cell division. For example, compositions useful for cancer therapy include, but are not limited to genes encoding tumor suppressor gene products/antigens antimetabolites, suicide gene products, anti-angiogenesis agents, immunostimulatory agents, and combinations thereof, as described further herein below. See generally Kirk & Mule, 2000; Mackensen et al., 1997; Walther & Stein, 1999; and references cited therein.

In one embodiment of the invention, labeled gene therapy constructs are used for cancer therapy. Angiogenesis and a suppressed immune response play central roles in the pathogenesis of malignant disease and tumor growth, invasion, and metastasis. Thus, therapeutic nucleic acids encode in one embodiment polypeptides, in another embodiment oligonucleotides, and in another embodiment peptide-nucleic acids having an ability to induce an immune response and/or an anti-angiogenic response in vivo.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation).

Representative therapeutic proteins with immunostimulatory effects include but are not limited to cytokines (e.g., IL-2, IL-4, IL-7, IL-12, interferons, granulocyte-macrophage colony-stimulating factor (GM-CSF), tumor necrosis factor alpha (TNF-α), immunomodulatory cell surface proteins (e.g., human leukocyte antigen (HLA proteins), co-stimulatory molecules, and tumor-associated antigens. See Kirk & Mule, 2000; Mackensen et al., 1997; Walther & Stein, 1999; and references cited therein.

The term “angiogenesis” refers to the process by which new blood vessels are formed. The term “anti-angiogenic response” and “anti-angiogenic activity” as used herein, each refer to a biological process wherein the formation of new blood vessels is inhibited.

Representative proteins with anti-angiogenic activities that can be used in accordance with the present invention include: thrombospondin I (Kosfeld & Frazier, 1993; Tolsma et al., 1993; Dameron et al., 1994), metallospondin proteins (Carpizo & Iruela-Arispo, 2000), class I interferons (Albini et al., 2000), IL-12 (Voest et al, 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Malone et al., 1990; Eijan et al., 1991; Woltering et al., 1991).

In one embodiment of the invention, an anti-angiogenic polypeptide comprises Tie-2, an endothelium-specific receptor tyrosine kinase (Lin et al., 1998b). Endogenous ligands are bound by ectopically expressed Tie-2, and signaling via the endogenous Tie-2 receptor to promote tumor growth is thereby blocked.

In another embodiment of the invention, an anti-angiogenic polypeptide comprises a soluble form of vascular endothelial growth factor (VEGF) receptor. In still another embodiment, an anti-angiogenic polypeptide comprises the Flk-1 receptor. The soluble VEGF receptors can function as dominant negative inhibitors of VEGF signaling and have been used to promote tumor regression. See Goldman et al., 1998; Takayama et al., 2000; Lin et al., 1998a; and PCT International Publication No. WO 00/37502.

A gene therapy construct used in accordance with the methods of the present invention can also encode a therapeutic gene that displays both immunostimulatory and anti-angiogenic activities, for example, IL-12 (Dias et al., 1998; and references cited herein below), interferon-α (O'Byrne et al., 2000, and references cited therein), or a chemokine (Nomura & Hasegawa, 2000, and references cited therein). In addition, a gene therapy construct can encode a gene product with immunostimulatory activity and a gene product having anti-angiogenic activity. See e.g., Narvaiza et al., 2000.

II.C. Promoters

A gene therapy construct of the invention can employ any suitable promoter, including both constitutive promoters, inducible promoters, and tissue-specific promoters. Representative inducible promoters include chemically regulated promoters (e.g., the tetracycline-inducible expression system, Gossen & Bujard, 1992; Gossen & Bujard, 1993; Gossen et al., 1995), a radiosensitive promoter (e.g., the egr-1 promoter, Weichselbaum et al., 1994; Joki et al., 1995; the E-selectin promoter, Hallahan et al., 1995a), and heat-responsive promoters (Csermely et al., 1998; Easton et al., 2000; Ohtsuka & Hata, 2000). Representative tissue-specific promoters include the CEA promoter, which is selectively expressed in cancer cells (Hauck & Stanners, 1995; Richards et al., 1995).

II.D. Vectors

The halogen-labeled gene therapy constructs of the present invention comprise vectors that facilitate transduction and expression of the gene therapy construct in a host cell. The particular vector employed in accordance with the disclosed methods is not intended to be a limitation of the methods for in vivo imaging of a gene therapy construct as disclosed herein.

The term “vector”, as used herein to refer to a gene therapy vector, refers to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors comprising nucleic acids include plasmids, cosmids, and viral vectors.

The term “vector” also includes non-nucleic acid compositions that can facilitate introduction of nucleic acids into a host cell, for example a liposome. As described further herein below, constructs comprising non-nucleic acid vectors are prepared by encapsulating or otherwise associating nucleic acids having nucleotide sequences that enable its replication in a host cell.

Any suitable vector for delivery of the gene therapy construct can be used including, but not limited to viruses, plasmids, water-oil emulsions, polyethylene imines, dendrimers, micelles, microcapsules, liposomes, and cationic lipids. Representative vectors that are amenable to the labeling and imaging methods disclosed herein include viral vectors, plasmids, and liposomes, each described further herein below. Where appropriate, two or more types of vectors can be used together. For example, a plasmid vector can be used in conjunction with liposomes. See e.g., U.S. Pat. No. 5,928,944.

Suitable methods for introduction of the vector into cells include direct injection into a cell or cell mass, particle-mediated gene transfer, hyper-velocity gene transfer, electroporation, DEAE-Dextran transfection, liposome-mediated transfection, viral infection, and combinations thereof. A delivery method is selected based considerations such as the vector type, the toxicity of the encoded gene, and the condition to be treated.

Viral Gene Therapy Vectors. Representative viruses for gene transfer include, but are not limited to adenoviruses (Zwiebel et al., 1998; Hitt & Graham, 2000; Silman & Fooks, 2000), adeno-associated virus (Halbert et al., 1995; Guha et al., 2000; Tal, 2000; Smith-Arica & Bartlett, 2001), herpes simplex virus (e.g. herpes simplex virus type 1) (Cunningham & Davison, 1993; Yeung & Tufaro, 2000; Latchman, 2001), RNA negative strand viruses (e.g., mumps virus) (Palese et al., 1996), parvovirus (Srivastava, 1994; Shaughnessy et al., 1996), Epstein-Barr virus (Delecluse & Hammerschmidt, 2000; Komaki & Vos, 2000), alphaviruses (e.g., Sindbis virus and Semliki virus) (Lundstrom, 1999; Wahlfors et al., 2000), baculovirus (Sandig et al., 1996; Sarkis et al., 2000), retroviruses (Cruz et al., 2000b; Cruz et al., 2000a), polyoma and papilloma viruses (Krauzewicz & Griffin, 2000), and varicella-zoster virus (Cohen & Seidel, 1993). Methods for preparation of viral vectors for gene therapy can be found in the above-cited sources, and references cited therein, among other places.

Viral vectors are in one embodiment replication-deficient. That is, they lack one or more functional genes required for their replication, which prevents their uncontrolled replication in vivo and avoids undesirable side effects of viral infection. In one embodiment, all of the viral genome is removed except for the minimum genomic elements required to package the viral genome incorporating the therapeutic gene into the viral coat or capsid. For example, it is desirable to delete all the viral genome except the Long Terminal Repeats (LTRs) or Invented Terminal Repeats (ITRs) and a packaging signal. In the case of adenoviruses, deletions are typically made in the E1 region and optionally in one or more of the E2, E3 and/or E4 regions. In the case of retroviruses, genes required for replication, such as env and/or gag/pol can be deleted. Deletion of sequences can be achieved using recombinant techniques, for example, involving digestion with appropriate restriction enzymes, followed by religation. Replication-competent self-limiting or self-destructing viral vectors can also be used.

Nucleic acid constructs of the invention can be incorporated into viral genomes by any suitable technique known in the art. Typically, such incorporation will be performed by ligating the construct into an appropriate restriction site in the genome of the virus.

Viral genomes can then be packaged into viral coats or capsids by any suitable procedure. In particular, any suitable packaging cell line can be used to generate viral vectors of the invention. These packaging lines complement the replication-deficient viral genomes of the invention, as they include, typically incorporated into their genomes, the genes which have been deleted from the replication-deficient genome. Thus, the use of packaging lines allows viral vectors of the invention to be generated in culture. For example, suitable packaging lines for retroviruses include derivatives of PA317 cells, ψ-2 cells, CRE cells, CRIP cells, E-86-GP cells, and 293GP cells. Line 293 cells can be used for adenoviruses and adeno-associated viruses. Neuroblastoma cells can be used for herpes simplex virus, e.g. herpes simplex virus type 1.

Plasmid Gene Therapy Vectors. A gene therapy construct of the present invention can also include a plasmid. Advantages of using plasmid vectors include low toxicity and relatively simple large-scale production. A major obstacle that has prevented the widespread application of plasmid DNA is its relative inefficiency in gene transduction. Electroporation has been used to effectively transport molecules including DNA into living cells in vitro (Neumann et al., 1982). Recent reports have demonstrated the use of electroporation in vivo, for example to enhance local efficiency of chemotherapeutic agents (Hofmann et al., 1999; Sersa et al., 2000).

Plasmid transfection efficiency in vivo encompasses a multitude of parameters, such as the amount of plasmid, time between plasmid injection and electroporation, temperature during electroporation, and electrode geometry and pulse parameters (field strength, pulse length, pulse sequence, etc.). The methods disclosed herein can be optimized for a particular application by methods known to one of skill in the art, and the present invention encompasses such variations. See e.g., Heller et al., 1996; Vicat et al., 2000; and Miklavcic et al., 1998.

Liposomes. The present invention also envisions the use of gene therapy constructs comprising liposomes. Representative liposomes include, but are not limited to cationic liposomes, optionally coated with polyethylene glycol (PEG) to reduce non-specific binding of serum proteins and to prolong circulation time. See Koning et al., 1999; Nam et al., 1999; and Kirpotin et al., 1997. Temperature-sensitive liposomes can also be used, for example THERMOSOMES™ as disclosed in U.S. Pat. No. 6,200,598. A gene therapy construct can further comprise plasmid-liposome complexes as described in U.S. Pat. No. 5,851,818.

Liposomes can also be prepared by any of a variety of techniques that are known in the art. See e.g., Betageri et al., 1993; Gregoriadis, 1993; Janoff, 1999; Lasic & Martin, 1995; Nabel, 1997; and U.S. Pat. Nos. 4,235,871; 4,551,482; 6,197,333; and 6,132,766. As one example, PEG 2000-PE, cholesterol, Dipalmitoyl phosphocholine (Avanti® Polar Lipids, Inc., Alabaster, Ala., United States of America), DiI (lipid fluorescent marker available from Molecular Probes, Inc., Eugene, Oreg., United States of America), and maleimide-PEG-2000-DOPE are dissolved in chloroform and mixed at a ratio of 10:43:43:2:2 in a round bottom flask as described in Leserman et al., 1980. The organic solvent is removed by evaporation followed by desiccation under vacuum for 2 hours. Liposomes are prepared by hydrating the dried lipid film in phosphate-buffered saline at a lipid concentration of 10 mM. The suspension is then sonicated 3×5 minutes until clear, forming unilamellar liposomes of 100 nm in diameter.

Entrapment of an active agent within liposomes can be carried out using any conventional method in the art. In preparing liposome compositions, stabilizers such as antioxidants and other additives can be used (Leserman, 1980; Betageri et al., 1993; Gregoriadis, 1993; Lasic & Martin, 1995; Nabel, 1997; Janoff, 1999).

Other lipid carriers can also be used in accordance with the claimed invention, such as lipid microparticles, micelles, sphingosomes, lipid suspensions, and lipid emulsions. See e.g., Labat-Moleur et al., 1996 and U.S. Pat. Nos. 5,011,634; 5,814,335; 6,056,938; 6,217886; 5,948,767; and 6,210,707.

III. In Vivo Detection of a Heterologous Antigen

The present invention further provides an antibody-based method for in vivo imaging of drug biodistribution following administration to a subject. The method comprises: (a) administering to a subject an effective dose of the drug, wherein the drug comprises a heterologous antigen; (b) administering to the subject an antibody that specifically binds the heterologous antigen, wherein the antibody comprises a label that can be detected in vivo; and (c) detecting the label in vivo, whereby the drug is detected in the subject.

The term “drug” as used herein refers to any substance having biological or detectable activity. Thus, the term “drug” includes therapeutic and diagnostic compositions. The term “drug” also includes any substance that is desirably delivered to a tumor. A drug can comprise a small molecule, a nucleic acid, a gene therapy construct (labeled or unlabeled embodiments described herein above), a polypeptide, an antibody or fragment thereof, a peptide, a polysaccharide, a lipid, and combinations thereof.

The term “antigen” includes any substance that can be specifically bound by an antibody molecule. Thus, the term “antigen” encompasses small molecules, nucleic acids, proteins, peptides, peptide mimetics, and any other molecule or compound that comprises an antigen for antibody recognition.

The term “heterologous antigen” as used herein refers to an antigen that originates from a source foreign to the intended host cell. Thus, a heterologous antigen is not present in a host cell of a subject in the absence of administration of the heterologous antigen to the subject. Alternatively stated, a heterologous antigen comprises an antigen other than an endogenous antigen. In one embodiment, a heterologous antigen is substantially inert or lacking metabolic or signaling activity in the subject. Thus, the term “heterologous antigen”, as used herein, further excludes antigens comprising a mutated form of an endogenous antigen as such mutate forms can still possess biological activity.

The term “endogenous antigen” as used herein refers to an antigen present in a host cell of a subject in the absence of introduction of the antigen by the hand of man.

In one embodiment of the invention, a drug composition to be administered to a subject comprises a heterologous antigen. The drug itself can comprise a heterologous antigen. In another embodiment, a drug can comprise a heterologous antigen that is conjugated to, encoded by, or otherwise associated with a drug.

Thus, an antibody that specifically recognizes a heterologous antigen shows substantially no binding to an endogenous antigen. The term “substantially no binding” encompasses non-specific binding and/or unsaturable binding. An antibody that shows substantially no binding to an endogenous antigen does not display specific and saturable binding as known in the art.

III.A. Peptide Antigens

In one embodiment of the invention, the heterologous antigen comprises a peptide. A peptide of the present invention has an amino acid sequence comprising in one embodiment at least about 3 residues, in another embodiment about 3 to about 50 residues, in another embodiment about 3 to about 20 residues, and in yet another embodiment about 3 to about 10 residues. Representative heterologous peptide antigens useful for detection methods of the present invention are set forth as SEQ ID NOs: 1 and 2. See Examples 5 and 6.

A heterologous peptide antigen of the present invention can be subject to various changes, substitutions, insertions, and/or deletions where such changes provide for certain advantages in its use. Thus, the term “peptide” encompasses any of a variety of forms of peptide derivatives, that include amides, conjugates with proteins, cyclone peptides, polymerized peptides, conservatively substituted variants, analogs, fragments, peptides, chemically modified peptides, and peptide mimetic. The term “heterologous peptide antigen” each refers to a peptide as defined herein above that comprises a peptide that is not naturally occurring in the intended host cell.

Peptides of the invention can comprise naturally occurring amino acids, synthetic amino acids, genetically encoded amino acids, non-genetically encoded amino acids, and combinations thereof. Peptides can include both L-form and D-form amino acids.

Representative non-genetically encoded amino acids include, but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amino hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The term “conservatively substituted variant” refers to a peptide having an amino acid residue sequence substantially identical to a sequence of a reference peptide antigen in which one or more residues have been conservatively substituted with a functionally similar residue and which displays the antigenicity and heterologous nature as described herein. The phrase “conservatively substituted variant” also includes peptides wherein a residue is replaced with a chemically derivatized residue, provided that the resulting peptide displays antigenicity and a heterologous nature as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Peptides of the present invention also include peptides having one or more additions and/or deletions or residues relative to the sequence of a peptide whose sequence is disclosed herein, so long as the requisite antigenicity and heterologous nature of the peptide is maintained. The term “fragment” refers to a peptide having an amino acid residue sequence shorter than that of a peptide disclosed herein.

Additional residues can also be added at either terminus of a peptide for the purpose of providing a “linker” by which peptides of the present invention can be conveniently affixed to a label or solid matrix, or carrier. Linkers will generally comprise at least one amino acid and can be 40 or more residues, more often 1 to 10 residues, but do alone not constitute heterologous peptide antigens. Typical amino acid residues used for linking are tyrosine, cysteine, lysine, glutamic and aspartic acid, or the like.

In addition, a peptide can be modified by terminal-NH₂ acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong half-life of the peptides in solutions, particularly biological fluids where proteases can be present.

Peptide cyclization is also a useful terminal modification, and is particularly preferred because of the stable structures formed by cyclization and in view of the biological activities observed for such cyclic peptides as described herein. An exemplary method for cyclizing peptides is described by Schneider & Eberle, 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxyl termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

The term “peptoid” is used herein to refer to a peptide wherein one or more of the peptide bonds are replaced by pseudopeptide bonds including, but not limited to a carba bond (CH₂-CH₂), a depsi bond (CO—O), a hydroxyethylene bond (CHOH—CH₂), a ketomethylene bond (CO—CH₂), a methylene-ocy bond (CH₂—O), a reduced bond (CH₂—NH), a thiomethylene bond (CH₂—S), an N-modified bond (—NRCO—), and a thiopeptide bond (CS—NH). See e.g. Corringer et al., 1993; Garbay-Jaureguiberry et al., 1992; Pavone et al., 1993; Tung et al., 1992; Urge et al., 1992.

Peptides of the present invention, including peptoids, can be synthesized by any of the techniques that are known to those skilled in the art of peptide synthesis. Synthetic chemistry techniques, such as a solid-phase Merrifield-type synthesis, are employed for reasons of purity, antigenic specificity, freedom from undesired side products, ease of production, and the like. A summary of representative techniques can be found in Stewart & Young, 1969; Merrifield, 1969; Fields & Noble, 1990; and Bodanszky, 1993. Solid phase synthesis techniques can be found in Andersson et al., 2000, references cited therein, and in U.S. Pat. Nos. 6,015,561; 6,015,881; 6,031,071; and 4,244,946. Peptide synthesis in solution is described by Schröder & Lübke, 1965. Appropriate protective groups usable in such synthesis are described in the above texts and in McOmie, 1973. Peptides, including peptides comprising non-genetically encoded amino acids, can also be produced in a cell-free translation system, such as described by Shimizu et al., 2001. In addition, peptides having a specified amino acid sequence can be purchased from commercial sources (e.g., Biopeptide Co., LLC, San Diego, Calif., United States of America and PeptidoGenics, Livermore, Calif., United States of America). In one embodiment of the invention, a heterologous peptide antigen is recombinantly produced as described further herein below.

The term “peptide mimetic” as used herein refers to a ligand that mimics the biological activity of a reference peptide, by substantially duplicating the antigenicity of the reference peptide, but it is not a peptide or peptoid. In one embodiment, a peptide mimetic has a molecular weight of less than about 700 daltons. A peptide mimetic can be designed or selected using methods known to one of skill in the art. See e.g., U.S. Pat. Nos. 5,811,392; 5,811,512; 5,578,629; 5,817,879; 5,817,757; and 5,811,515.

Any peptide or peptide mimetic of the present invention can be used in the form of a pharmaceutically acceptable salt. Suitable acids which are capable of the peptides with the peptides of the present invention include, but are not limited to inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. In one embodiment, a pharmaceutically acceptable salt is a HCl salt. In another embodiment, a pharmaceutically acceptable salt is a TFA salt.

Suitable bases capable of forming salts with the peptides of the present invention include inorganic bases such as sodium hydroxide, ammonium hydroxide, potassium hydroxide and the like; and organic bases such as mono-, di-, and tri-alkyl and aryl amines (e.g. triethylamine, diisopropyl amine, methyl amine, dimethyl amine and the like), and optionally substituted ethanolamines (e.g. ethanolamine, diethanolamine and the like).

III.B. Preparation of a Drug Comprising a Heterologous Antigen

The detection methods of the present invention rely on a predictable association between a heterologous antigen and a drug, such that the detectable presence of the heterologous antigen is indicative of the drug distribution. Such an association can be created by, for example, conjugation of a heterologous antigen to a drug. Alternatively or in addition, a heterologous peptide antigen can be recombinantly expressed, such that its detectable expression is indicative of the expression of an encoded therapeutic gene product.

A heterologous antigen, including nucleic acid, peptide, and small molecule antigens, can be coupled to drugs or drug carriers using methods known in the art including, but not limited to carbodiimide conjugation, esterification, sodium periodate oxidation followed by reductive alkylation, and glutaraldehyde crosslinking. Protocols for performing such conjugation methods can be found, for example, in Goldman et al., 1997; Cheng, 1996; Neri et al., 1997; Nabel, 1997; Park et al., 1997; Pasqualini et al., 1997; Bauminger & Wilchek, 1980; U.S. Pat. No. 6,071,890; and European Patent No. 0 439 095.

When a therapeutic composition of the invention comprises a gene therapy construct, a heterologous antigen can comprise an antigen that is conjugated or otherwise associated with the gene therapy vector, as described herein above. Alternatively, a heterologous antigen can comprise a peptide encoded by nucleotide sequences of the gene therapy construct. Recombinant expression of a heterologous peptide antigen can be variably accomplished by employing any suitable construct design, representative approaches being described herein below.

A heterologous antigen that is encoded by a gene therapy construct can be expressed under the direction of any suitable promoter, including constitutive promoters, inducible promoters, and tissue-specific promoters. Representative inducible promoters include chemically regulated promoters (e.g., the tetracycline-inducible expression system, Gossen & Bujard, 1992; Gossen & Bujard, 1993; Gossen et al., 1995), a radiosensitive promoter (e.g., the egr-1 promoter, Weichselbaum et al., 1994; Joki et al., 1995; the E-selectin promoter, Hallahan et al., 1995a), and heat-responsive promoters (Csermely et al., 1998; Easton et al., 2000; Ohtsuka & Hata, 2000). Representative tissue-specific promoters include the CEA promoter, which is selectively expressed in cancer cells (Hauck & Stanners, 1995; Richards et al., 1995).

In one embodiment of the invention, nucleotide sequences encoding a therapeutic molecule and a heterologous peptide antigen are separate open reading frames within a single gene therapy construct. In this case, the nucleotide sequences encoding the therapeutic molecule and nucleotide sequences encoding the heterologous peptide antigen are co-expressed in the same cell, but each of the encoded therapeutic molecule and the heterologous peptide antigen is free of the other.

Co-expression can be directed by separate promoters, although co-expression can also be directed by duplicate inclusion of a same promoter sequence. Thus, in one embodiment, co-expression is directed by a single promoter. For example, nucleotide sequences encoding a therapeutic molecule and a heterologous peptide antigen can be cloned into a bi-cistronic vector that simultaneously directs transcription of each sequence using a single promoter. A bi-cistronic vector can include an internal ribosome entry site (IRES) derived from any suitable source, including an IRES sequence derived from a cellular or viral genome. Representative IRES sequences and methods for construct design employing the same can be found in Klump et al, 2001; Hennecke et al., 2001; Furler et al., 2001; Harries et al., 2000; Chappell et al., 2000; Attal et al., 1999; Jespersen et al., 1999; Havenga et al., 1998; and references cited therein, among other places.

In another embodiment, a nucleotide sequence encoding a therapeutic molecule and a nucleotide sequence encoding a heterologous antigen can be included in separate gene therapy vectors that are combined in a single therapeutic composition. For example, a therapeutic composition of the present invention can comprise a first vector that directs expression of a therapeutic molecule in admixture with a second vector that directs expression of a heterologous label. In one embodiment, the first vector and second vector are a same type of vector (e.g., both vectors are plasmids or both vectors are adenovirus). In another embodiment, the first vector and second vector comprise substantially identical nucleotide sequences other than the nucleotide sequences encoding the therapeutic molecule and label peptide/label polypeptide, such that expression of the first vector and the second vector is substantially similar. The term “substantially similar”, as used herein to describe gene expression, refers to a degree of coincidence and level of expression such that expression of the heterologous antigen is indicative of expression of the therapeutic molecule.

In one embodiment of the invention, a nucleotide sequence that encodes a heterologous peptide antigen is operatively linked to a gene encoding a therapeutic polypeptide or therapeutic oligonucleotide such that the resulting therapeutic molecule is fused to the heterologous peptide antigen. For example, an encoded therapeutic polypeptide can be an elongated polypeptide, wherein the heterologous peptide antigen is included at the amino terminus or at the carboxyl terminus of the therapeutic polypeptide. Alternatively or in addition, the heterologous peptide antigen can be included as a non-terminal addition to the therapeutic polypeptide. See Examples 5 and 6.

A gene therapy construct that includes sequences for recombinant expression of a heterologous peptide antigen can further comprise a nucleotide sequence that encodes a signal peptide for secretion or membrane localization of the heterologous peptide antigen. The terms “membrane localization” is used to refer to presentation of the heterologous peptide antigen at the extra cellular surface of a transduced cell. Thus, membrane localization encompasses insertion in a cell membrane, tethering to a cell membrane via a membranous anchor, and/or any other association with the cell membrane such that the heterologous peptide is substantially accessible for binding to an administered antibody. For example, a genetically encoded heterologous peptide antigen can be targeted to the cell surface by fusion to a peptide signal/membrane anchoring domain (see e.g., Simonova et al., 1999). Membrane localization can also be mediated by targeting domains that bind to lipid ligands embedded in the cell membrane, for example a pleckstrin homology domain, a protein kinase C homology-1 or -2 domain, or a FYVE domain (Hurley & Misra, 2000; Johnson et al., 2000; Lemmon & Ferguson, 2000).

III.C. Antibodies that Recognize a Heterologous Antigen

Thus, the present invention further provides a composition for imaging of drug distribution. In one embodiment, the composition comprises (1) an antibody that specifically recognizes a drug comprising a heterologous antigen; or (2) an antibody that specifically recognizes a heterologous antigen conjugated to, encoded by, or otherwise associated with the drug.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). In one embodiment, an antibody of the invention is a monoclonal antibody.

The terms “antigen binding site” and “functional portion”, as used herein to describe an antibody, each refer to the part of the antibody that binds a heterologous antigen.

Techniques for preparing and characterizing antibodies are known in the art. See e.g., Harlow & Lane, 1988; and U.S. Pat. Nos. 4,196,265; 4,946,778; 5,091,513; 5,132,405; 5,260,203; 5,677,427; 5,892,019; 5,985,279; 6,054561).

An antibody of the invention can further be mutagenized or otherwise modified to preferably improve antigen binding and/or antibody stability. For example, to prevent undesirable disulfide bond formation, a nucleotide sequence encoding the variable domain of an antibody or antibody fragment can be modified to eliminate at least one of each pair of codons that encode cysteines for disulfide bond formation. Recombinant expression of the modified nucleotide sequence, for example in a prokaryotic expression system, results in an antibody having improved stability. See U.S. Pat. No. 5,854,027.

Methods for conjugating a detectable label to an antibody preferably do not disrupt the antigen binding site. Representative labeling methods are described herein below under the heading In Vivo Imaging of Drug Biodistribution. Labeled antibodies can be lyophilized and stored until use as described in U.S. Pat. No. 6,080,384, among other places.

A labeled antibody for use in the methods of the present invention is administered to a subject in any suitable manner. In one embodiment, an antibody is administered by parenteral injection, or more preferably, by intravascular injection. Administration routes and dose are described further herein below. As one example, 2.0 mg of a polyclonal antibody labeled with 1.2 mCi/MEq of ¹¹¹indium can comprise a diagnostic amount when administered to a human subject (Datz et al., 1994). Humanized antibodies or human monoclonal antibodies can be administered to a subject at a dose of up to 200 mg per week (U.S. Pat. No. 5,965,106).

IV. Preparation and Administration of a Therapeutic and/or Diagnostic Composition

IV.A. Drug Carriers

Halogen-labeled gene therapy constructs and drugs comprising a heterologous antigen, as disclosed herein, can further comprise a drug carrier to facilitate drug preparation and administration. Any suitable drug delivery vehicle or carrier can be used including, but not limited to a gene therapy vector (described herein above), a nanosphere (Manome et al., 1994; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a porous or aerodynamically light particle (U.S. Pat. Nos. 6,254,854 and 6,136,295), a nanosuspension (U.S. Pat. No. 5,858,410), a polymeric micelle or conjugate (Goldman et al., 1997) and U.S. Pat. Nos. 4,551,482, 5,714,166, 5,510,103, 5,490,840, and 5,855,900), and a polysome (U.S. Pat. No. 5,922,545).

The present invention also encompasses adoptive therapy wherein cells are administered to a host with the aim that the cells mediate in vivo biological activities, such as localization to tumor sites and stimulation of host cell responses. Thus, a drug carrier of the present invention can comprise cells transformed with a halogen-labeled gene therapy construct as disclosed herein. Such cells can be used for ex vivo, in vivo, and in vitro gene transfer (e.g. Yang, 1992, and references cited therein). Representative cell types are employed on adoptive therapy include but are not limited to leukocytes, and endothelial progenitor cells.

The cells can be of any type that is compatible with the recipient's immune system. As with any transplantation of cells or tissue, the major tissue transplantation antigens of the administered cells will match the major tissue transplantation antigens of the recipient's cells. In one embodiment, the cells administered are derived from a tumor of the intended recipient, e.g. tumor cells can be removed from the intended recipient, transformed or transfected as appropriate then returned, in order to effect cell therapy. In another embodiment, cells can be derived from other individuals with compatible tissue transplantation antigens, such as close relatives. In another embodiment, cells can be HLA matched cells, e.g. HLA matched fibroblasts, which do not give rise to adverse immune reaction.

When cells are to be modified for the purpose of ex vivo gene transfer, vectors disclosed herein can be introduced into cells (e.g., human primary or secondary cells such as fibroblasts, epithelial cells including mammary and intestinal epithelial cells, endothelial cells, blood components including lymphocytes and bone marrow cells, glial cells, hepatocytes, keratinocytes, muscle cells neural cells, or the precursors of these or any other malignant cell types; non-human animal cells; and other eukaryotic cells) by standard methods of transfection, as described herein above.

IV.B. Targeting Ligands

The term “target cell” as used herein refers to a cell intended to be treated by a therapeutic agent. A target cell is in one embodiment a cell derived from a subject in need of therapeutic treatment. For example, a tumor cell and a cancer cell are target cells for cancer treatment.

As desired, compositions of the present invention can include a targeting or homing molecule that facilitates delivery of a drug to an intended in vivo site. A targeting molecule can comprise, for example, a ligand that shows specific affinity for a target molecule in the target tissue. See U.S. Pat. Nos. 6,068,829 and 6,232,287. A targeting molecule can also comprise a structural design that mediates tissue-specific localization. For example, extended polymeric molecules can be conjugated to drugs to mediate tumor localization. See U.S. Pat. No. 5,762,909.

Targeting molecules that mediate localization to tumors include in one embodiment ligands that show specific binding to antigens present on tumor vasculature, tumor endothelium (e.g., endothelial cells associated with tumor vasculature), or on tumor cells. For example, a targeting ligand can comprise an antibody or antibody fragment that specifically binds a tumor marker such as Her2/neu (v-erb-b2 avian erythroblastic leukemia viral oncogene homologue 2), CEA (carcinoembryonic antigen), or a ferritin receptor, or that specifically binds to a marker associated with tumor vasculature (integrins, tissue factor, or β-fibronectin isoform). Alternatively, a targeting ligand can comprise a peptide or peptide mimetic that behaves as a tumor homing molecule (Wickham et al., 1995; Staba et al., 2000; International Publication Nos. WO 98/10795 and WO 01/09611; and U.S. Pat. No. 6,180,084).

In one embodiment of the invention, a therapeutic composition comprises a targeting ligand that selectively binds a radiation-induced tumor target. Such a composition can be used in accordance with methods for x-ray-guided drug delivery (U.S. Pat. No. 6,159,443). Briefly, the method includes the steps of: (a) administering to a subject a therapeutic and/or diagnostic agent comprising a ligand that binds a radiation-inducible molecule; and (b) irradiating a tumor in the subject, whereby the drug is delivered to the tumor.

The term “induce”, as used herein to refer to changes resulting from radiation exposure, encompasses activation of gene transcription or regulated release of proteins from cellular storage reservoirs to vascular endothelium. Alternatively, induction can refer to a process of conformational change, also called activation, such as that displayed by the GPIIb/IIIa integrin receptor upon radiation exposure (Staba et al., 2000; Hallahan et al., 2001). See also U.S. Pat. No. 6,159,443. Irradiated tumors can be targeted using antibodies, peptides, or small molecules that specifically recognize radiation-induced surface proteins as disclosed in Hallahan et al., 2001; Staba et al., 2000; and U.S. Pat. No. 6,159,443.

Targeting ligands can be coupled to drugs or drug carriers using methods known in the art. See e.g., Cheng, 1996; Kirpotin et al., 1997; Nabel, 1997; Neri et al., 1997; Park et al., 1997; Pasqualini et al., 1997; U.S. Pat. No. 6,071,890; and European Patent No. 0 439 095. Alternatively, pseudotyping of a retrovirus can be used to target a virus towards a particular cell (Marin et al., 1997). In one embodiment, the targeting method preserves the activity of the therapeutic composition. In another embodiment, a composition comprising an inducible therapeutic agent is used. For example, a targeting ligand and therapeutic composition can be conjugated using a selectively hydrolyzable bond, such as an acid-labile or enzyme-sensitive bond. See U.S. Pat. No. 5,762,918.

IV.C. Formulation

A therapeutic composition, a diagnostic composition, or a combination thereof, of the present invention comprises in one embodiment a pharmaceutical composition that includes a pharmaceutically acceptable carrier. Suitable formulations include aqueous and non-aqueous sterile injection solutions which can contain anti-oxidants, buffers, bacteriostats, bactericidal antibiotics, and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some non-limiting ingredients are SDS, in one embodiment in the range of 0.1 to 10 mg/ml, and in another embodiment about 2.0 mg/ml; and/or mannitol or another sugar, in one embodiment in the range of 10 to 100 mg/ml, and in another embodiment about 30 mg/ml; and/or phosphate-buffered saline (PBS). Any other agents conventional in the art having regard to the type of formulation in question can be used.

The therapeutic regimens and pharmaceutical compositions of the invention can be used with additional adjuvants or biological response modifiers including, but not limited to, the cytokines IFN-α, IFN-γ, IL-2, IL-4, IL-6, TNF, or other cytokine affecting immune cells.

IV.D. Administration

Suitable methods for administration of a therapeutic composition, a diagnostic composition, or combination thereof, of the present invention include, but are not limited to systemic administration, parenteral administration (including, but not limited to intravascular, intramuscular, intraarterial administration), oral delivery, subcutaneous administration, inhalation, intratracheal installation, surgical implantation, transdermal delivery, local injection, and hyper-velocity injection/bombardment. Where applicable, continuous infusion can enhance drug accumulation at a target site. See e.g., U.S. Pat. No. 6,180,082.

The particular mode of drug administration of the present invention depends on various factors including, but not limited to the distribution and abundance of cells to be treated, the vector and/or drug carrier employed, additional tissue- or cell-targeting features, and mechanisms for metabolism or removal of the drug from its site of administration.

The administration method can further include treatments for enhancing drug delivery. For example, electromagnetic waves or ultrasonic radiation can be used to enhance drug delivery in solid tumors. See U.S. Pat. No. 6,165,440. Heating of the particles or movement of the particles in response to ultrasonic waves results in perforation of tumor blood vessels, microconvection in the interstitium, and perforation of cancer cell membranes, thereby facilitating movement of intravascularly administered drugs to tumor cells. See also, U.S. Pat. No. 6,234,990. Other methods include ionotophoresis (U.S. Pat. Nos. 6,001,088; 5,499,971), electroporation (U.S. Pat. No. 6,041,253), electromagnetic field generation by ultra-wide band short pulses (U.S. Pat. No. 6,261,831), and hormone treatment (U.S. Pat. No. 5,962,667). Also included are treatments that facilitate targeting of a targeting ligand. For example, drug administration can further include radiotherapy for x-ray-guided drug delivery, as described herein above. See also U.S. Pat. No. 6,159,443 and Hallahan et al., 2001.

The administration method can also include treatments for drug release or drug activation. For example, a composition comprising a therapeutic agent conjugated to a drug carrier of targeting molecule via a selectively hydrolyzable bond can be released by local provision of a hydrolyzing agent (U.S. Pat. No. 5,762,918). In the case of a gene therapy construct, gene expression of a therapeutic polypeptide or therapeutic oligonucleotide can be regulated using an inducible promoter. Useful promoters for this purpose include constructs that are transcriptionally activated by small molecules such as tetracycline (Deuschle et al., 1995; Gossen et al., 1995) and hormones (No et al., 1996; Abruzzese et al., 1999; Burcin et al., 1999). Also included are radiation-inducible constructs, such as those employing the Egr-1 promoter or NF-κB promoter (Weichselbaum et al., 1991; Weichselbaum et al., 1994). A heat-inducible construct can also be used to direct gene transcription in response to local hyperthermia (Madio et al, 1998; Gerner et al., 2000; Vekris et al., 2000).

Similarly, the administration method employed can include treatments that augment drug efficacy. For example, in vivo electroporation and electromagnetic field generation can enhance the potency of chemotherapeutic drugs (Hofmann et al., 1999; Sersa et al., 2000; U.S. Pat. No. 6,261,831). As another example, radiotherapy can add to or potentiate the effect of some anti-angiogenic drugs. See e.g., Griscell et al., 2000; Fabbro et al., 2000.

IV.E. Dose

For therapeutic applications, a therapeutically effective amount of a composition of the invention is administered to a subject. A “therapeutically effective amount” is an amount of the therapeutic composition sufficient to produce a measurable biological response (including, but not limited to an immunostimulatory response, an anti-angiogenic response, a cytotoxic response, or tumor regression). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including, but not limited to the activity of the therapeutic composition, formulation, the route of administration, combination with other drugs or treatments, severity of the condition being treated (e.g., in the case of a tumor, tumor size and longevity), and the physical condition and prior medical history of the subject being treated. In one embodiment, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For diagnostic applications, a detectable amount of a composition of the invention is administered to a subject. A “detectable amount”, as used herein to refer to a diagnostic composition, refers to a dose of such a composition that the presence of the composition can be determined in vivo or in vitro. A detectable amount will vary according to a variety of factors, including, but not limited to chemical features of the drug being labeled, the detectable label, labeling methods, the method of imaging and parameters related thereto, metabolism of the labeled drug in the subject, the stability of the label (e.g. the half-life of a radionuclide label), the time elapsed following administration of the drug and/or labeled antibody prior to imaging, the route of drug administration, and the physical condition and prior medical history of the subject. Thus, a detectable amount can vary and can be tailored to a particular application. After study of the present disclosure, and in particular the Examples, it is within the skill of one in the art to determine such a detectable amount.

For local administration of viral vectors, previous clinical studies have demonstrated that up to 10¹³ pfu of virus can be injected with minimal toxicity. In human patients, 1×10⁹-1×10¹³ pfu are routinely used. See Habib et al., 1999. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose can also be modified to maximize anti-tumor and/or anti-angiogenic activity. Representative criteria and methods for assessing anti-tumor and/or anti-angiogenic activity are described herein below.

For administration of therapeutic and/or diagnostic compositions comprising a small molecule, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses can also given in milligrams per square meter (mg/m²) of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m². See also U.S. Pat. Nos. 5,326,902 and 5,234,933, and PCT International Publication No. WO 93/25521.

For the purposes of cell therapy, cells (e.g. cells for ex vivo therapy) can be delivered by intradermal administration in one embodiment and by subcutaneous administration in another embodiment. A person of skill in the art will be able to choose an appropriate dosage, e.g. the number and concentration of cells, to take into account the fact that only a limited volume of fluid can be administered in this manner.

V. In Vivo Imaging of Drug Biodistribution

The present invention provides halogen-labeled gene therapy constructs that can be directly detected using the methods for imaging described herein below. The present invention also provides drugs comprising a heterologous antigen, which can be detected using an antibody comprising a detectable label. Following administration of the labeled gene therapy construct or antibody to a subject, and after a time sufficient for binding, the biodistribution of the composition can be visualized. The term “time sufficient for binding” refers to a temporal duration that permits binding of the labeled agent to a heterologous antigen. In one embodiment, the detectable label can be detected in vivo.

The term “in vivo”, as used herein to describe imaging or detection methods, refers to generally non-invasive methods such as scintigraphic methods, magnetic resonance imaging, ultrasound, and fluorescence, each described briefly herein below. The term “non-invasive methods” does not exclude methods employing administration of a contrast agent to facilitate in vivo imaging.

In one embodiment of the invention, SPECT imaging is in combination with CT imaging. CT/SPECT (HAWKEYE™ model available from GE Medical Systems, Waukesha, Wis., United States of America) is an imaging modality that sequentially acquires data from computerized tomography and single photon emission tomography. This technology was developed and validated by Vanderbilt University (Nashville, Tenn., United States of America) and GE Medical Systems (Waukesha, Wis., United States of America). The CT scan provides the anatomical information such as which organ system contains the radiotracer. The SPECT scan is used to detect the radiotracer within the animal model.

In another embodiment of the invention, the disclosed methods for labeling and in vivo imaging are used in combination. For example, a halogen-labeled gene therapy construct can further comprise a nucleotide sequence that encodes a heterologous antigen. Expression of the gene therapy vector can be detected using a labeled antibody that specifically recognizes the heterologous peptide antigen as disclosed herein. In one embodiment, the antibody comprises a detectable label that can be detected in vivo and that is other than a scintigraphic label. Thus, the present invention provides methods and compositions for the simultaneous detection of a gene therapy construct and recombinant gene expression. For example, the distribution of the gene therapy construct could be detected using scintigraphic imaging, and the sites of gene expression could be assayed using magnetic resonance imaging.

V.A. Scintigraphic Imaging

Scintigraphic imaging methods include SPECT (Single Photon Emission Computed Tomography), PET (Positron Emission Tomography), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions.

A representative method for SPECT imaging is described in Example 2. Other imaging instruments suitable for practicing the method of the present invention, and instruction for using the same, are readily available from commercial sources. Both PET and SPECT systems are offered by ADAC (Milpitas, Calif., United States of America) and Siemens (Hoffman Estates, Ill., United States of America. Related devices for scintigraphic imaging can also be used, such as a radio-imaging device that includes a plurality of sensors with collimating structures having a common source focus.

When scintigraphic imaging is employed, the detectable label comprises in one embodiment a radionuclide label, and in another embodiment a radionuclide label selected from the group consisting of ¹⁸fluorine, ⁶⁴copper, 65copper, ⁶⁷gallium, 68gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶ iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, ¹⁶⁸thulium, and nitride or oxide forms derived there from. In one embodiment of the invention, the radionuclide label comprises ¹⁸fluorine, ¹²³iodine, ¹²⁵iodine, or ¹³¹iodine.

Methods for radionuclide-labeling of a molecule so as to be used in accordance with the disclosed methods are known in the art. For example, a targeting molecule can be derivatized so that a radioisotope can be bound directly to it (Yoo et al., 1997). For example, β-mercaptoethanol can be used to reduce disulfide bonds to sulfhydryl groups capable of binding to ^{99m}Tc. Alternatively, a linker can be added that to enable conjugation. Representative linkers include diethylenetriamine pentaacetate (DTPA)-isothiocyanate and succinimidyl 6-hydrazinium nicotinate hydrochloride (SHNH) (U.S. Pat. Nos. 4,652,440 and 6,024,938). Labeling can also be accomplished by reduction of radionuclides to enable binding to antibodies comprising at least one disulfide group, and preferably multiple adjacent free sulfhydryl groups (U.S. Pat. Nos. 5,328,679 and 6,080,384). See also U.S. Pat. Nos. 5,080,883; 5,047,227; and 4,671,958.

When the labeling moiety is a radionuclide, stabilizers to prevent or minimize radiolytic damage, such as ascorbic acid, gentisic acid, or other appropriate antioxidants, can be added to the composition comprising the labeled molecule.

V.B. Magnetic Resonance Imaging (MRI)

Magnetic resonance image-based techniques create images based on the relative relaxation rates of water protons in unique chemical environments. As used herein, the term “magnetic resonance imaging” refers to magnetic source techniques including convention magnetic resonance imaging, magnetization transfer imaging (MTI), proton magnetic resonance spectroscopy (MRS), diffusion-weighted imaging (DWI) and functional MR imaging (fMRI). See Rovaris et al. (2001) J Neurol Sci 186 Suppl 1:S3-9; Pomper & Port (2000) Magn Reson Imaging Clin N Am 8:691-713; and references cited therein.

Contrast agents for magnetic source imaging include but are not limited to paramagnetic or superparamagnetic ions, iron oxide particles (Weissleder et al., 1992; Shen et al., 1993), and water soluble contrast agents. Paramagnetic and superparamagnetic ions can be selected from the group of metals including iron, copper, manganese, chromium, erbium, europium, dysprosium, holmium and gadolinium. Representative metals are iron, manganese, and gadolinium. In one embodiment, a metal is gadolinium.

Those skilled in the art of diagnostic labeling recognize that metal ions can be bound by chelating moieties, which in turn can be conjugated to a therapeutic agent in accordance with the methods of the present invention. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Magnetic crystals suitable for imaging studies can also be loaded into matrix particles or coated (e.g. silanized), and the matrix particles or coated crystals are then coupled to an antibody (U.S. Pat. Nos. 5,597,531 and 5,736,349).

Images derived used a magnetic source can be acquired using, for example, a superconducting quantum interference device magnetometer (SQUID, available with instruction from Quantum Design, San Diego, Calif., United States of America). See U.S. Pat. No. 5,738,837.

V.C. Ultrasound

Ultrasound imaging can be used to obtain quantitative and structural information of a target tissue, including a tumor. Administration of a contrast agent, such as gas microbubbles, can enhance visualization of the target tissue during an ultrasound examination. Representative agents for providing microbubbles in vivo include, but are not limited to gas-filled lipophilic or lipid-based bubbles. See e.g., U.S. Pat. Nos. 6,245,318; 6,231,834; 6,221,018; and 5,088,499. In addition, gas or liquid can be entrapped in porous inorganic particles that facilitate microbubble release upon delivery to a subject. See e.g., U.S. Pat. Nos. 6,254,852 and 5,147,631.

Gases, liquids, and combinations thereof suitable for use with the invention include, but are not limited to air; nitrogen; oxygen; is carbon dioxide; hydrogen; nitrous oxide; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as tetramethylsilane; a low molecular weight hydrocarbon (e.g. containing up to 7 carbon atoms), for example an alkane such as methane, ethane, a propane, a butane or a pentane, a cycloalkane such as cyclobutane or cyclopentane, an alkene such as propene or a butene, or an alkyne such as acetylene; an ether; a ketone; an ester; a halogenated low molecular weight hydrocarbon (e.g. containing up to 7 carbon atoms); or a mixture of any of the foregoing. Halogenated hydrocarbon gases can show extended longevity, and thus are preferred for some applications. Representative gases of this group include, but are not limited to decafluorobutane, octafluorocyclobutane, decafluoroisobutane, octafluoropropane, octafluorocyclopropane, dodecafluoropentane, decafluorocyclopentane, decafluoroisopentane, perfluoropexane, perfluorocyclohexane, perfluoroisohexane, sulfur hexafluoride, and perfluorooctaines, perfluorononanes; perfluorodecanes, optionally brominated.

Attachment of lipophilic bubbles to antibodies can be accomplished via chemical crosslinking agents in accordance with standard protein-polymer or protein-lipid attachment methods (e.g., via 1-ethyl-3-(3-dimethylaminopropyl)-carbodiimide (EDC) or succinimidyl 3-(2-pyridyidithio)propionate (SPDP)). To facilitate antibody binding to a heterologous antigen, large gas-filled bubbles can be coupled to an antibody using a flexible spacer arm, such as a branched or linear synthetic polymer. See e.g. U.S. Pat. No. 6,245,318. An antibody can also be attached to porous inorganic particles by coating, adsorbing, layering, or reacting the outside surface of the particle with the antibody. See e.g. U.S. Pat. No. 6,254,852.

A description of ultrasound equipment and technical methods for acquiring an ultrasound dataset can be found in Coatney, 2001; Lees, 2001; and references cited therein.

V.D. Fluorescence

Non-invasive imaging methods can also comprise detection of a fluorescent label. An antibody conjugated to or otherwise associated with a lipophilic component (for example, a therapeutic agent, diagnostic agent, vector, or drug carrier) can be labeled with any one of a variety of lipophilic dyes that are suitable for in vivo imaging. See e.g. Fraser, 1996; Ragnarson et al., 1992; and Heredia et al., 1991. Representative labels include, but are not limited to carbocyanine and aminostyryl dyes, long chain dialkyl carbocyanines (e.g., DiI, DiO, and DiD available from Molecular Probes Inc., Eugene, Oreg., United States of America), and dialkylaminostyryl dyes. Lipophilic fluorescent labels can be incorporated using methods known to one of skill in the art. For example VYBRANT™ cell labeling solutions are effective for labeling of cultured cells of other lipophilic components (Molecular Probes Inc., Eugene, Oreg., United States of America).

A fluorescent label can also comprise sulfonated cyanine dyes, including Cy5.5 and Cy5 (available from Amersham Biosciences Corp., Piscataway, N.J., United States of America), IRD41 and IRD700 (available from Li-Cor, Inc., Lincoln, Nebr., United States of America), NIR-1 (available from Dejindo, Kumamoto, Japan), and LaJolla Blue (available from Diatron, Miami, Fla., United States of America). See also Licha et al., 2000; Weissleder et al., 1999; and Vinogradov et al., 1996.

In addition, a fluorescent label can comprise an organic chelate derived from lanthanide ions, for example fluorescent chelates of terbium and europium. See U.S. Pat. No. 5,928,627. Such labels can be conjugated or covalently linked to an antibody as disclosed therein.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

### EXAMPLES

The following Examples have been included to illustrate modes of the invention. Certain aspects of the following Examples are described in terms of techniques and procedures found or contemplated by the present inventor to work well in the practice of the invention. These Examples illustrate standard laboratory practices of the inventor. In light of the present disclosure and the general level of skill in the art, those of skill will appreciate that the following Examples are intended to be exemplary only and that numerous changes, modifications, and alterations can be employed without departing from the scope of the invention.

### Example 1

Radiohalogen-Labeled Adenovirus

Adenovirus vectors were prepared essentially as described by Hallahan et al., 1995b. Briefly, adenoviruses were produced in 293 cells that contain modified adenovirus genes that enable replication of the vector. Packaging 293 cells were supplemented with ¹³¹IUdR in complete medium. Polycarbonate tubes coated with 50 μg of 5-Tributylstannyl-2-deoxyuridine (Sn-UdR) were provided by Dr. A. I. Kassis (Harvard Medical School, Boston, Mass., United States of America). Dulbecco's 0.1 M phosphate-buffered saline (pH 7.3, 70 μl) was added to Sn-UdR-coated tubes with one IODO-BEAD® carrier (Pierce Chemical Co., Rockville, Ill., United States of America). Na¹³¹I (9 mCi in 10 μl of 0.1 N NaOH) was added with shaking for 1 minute at room temperature. The reaction mixture was withdrawn and IODO-BEAD® carriers were washed with H₂O. The HPLC profile of recovered ¹³¹IUdR compared to free ¹³¹I demonstrated 98% labeling efficiency.

Radiohalogen-labeled adenovirus encoding ExFlk.6His and ExTex.Strep were isolated using a CsCl density gradient (Hallahan et al., 1995b). Radiohalogen-labeled adenovirus vector was typically observed to contain about 90 μCi, which is a ratio of 1 mCi ¹³¹IUdR incorporated into vector DNA for every 1000 mCi ¹³¹IUdR provided in cell culture (0.1% labeling efficiency). The specific activity of the radiohalogenated vector is not considered to be a limitation of the present invention, so long as sufficient radiohalogen is incorporated to enable in vivo detection.

Although 90 μCi was sufficient to image vectors in 2 rats during a period of 2 days, improved labeling efficiency permits imaging during a longer temporal interval. Techniques for improving the specific activity of vectors are disclosed herein and can be performed by one of ordinary skill in the art.

For optimization of radiohalogen labeling of gene therapy vectors, separate packaging lines are used to evaluate labeling efficiency when variable labeling steps are employed, including but not limited to: (1) using thymidine-depleted medium; (2) pulse labeling with ¹³¹IUdR and other purine and pyrimidine analogues; (3) reducing medium volume; (4) increasing the density of 293 cells transduced with the vector, optionally to confluence; (5) increasing albumin concentration; (6) culturing 293 cells in glass dishes; (7) prolonging ¹³¹IUdR incubation time; and (8) adding ¹³¹IUdR overnight prior to vector transduction. The specific activity of a radiohalogenated vector is measured, and each variable technique that improves labeling efficiency is incorporated into the labeling protocol.

### Example 2

CT/SPECT Imaging of Radiohalogen-Labeled Adenovirus

¹³¹I-Ad.ExFlk (an adenovirus vector encoding ExFlk) was administered to tumor bearing rats by tail vein injection (negative control) or by intratumoral injection (positive control).

For animal imaging experiments, a dual-head gamma camera capable of single photon emission computed tomography was modified with the addition of an integrated x-ray transmission system for unambiguous radiotracer localization and attenuation compensation. To provide attenuation maps and anatomical localization, an x-ray tube and linear detector array were installed on a dual-head scintillation camera with 140 keV to 511 keV imaging capability.

The scintillation camera (MILLENIUM™ VG—Variable Geometry model available from General Electric Medical Systems, Milwaukee, Wis., United States of America) was equipped with ⅝ inch (15.9 mm) thick NaI(TI) crystals and a slip ring gantry permitting data acquisition while the detectors rotate around the subject. The x-ray tube operated in a continuous output mode, which was selectable up to a maximum of 140 kilovolt peak (kVp) at 2.5 milliamps (mA). The detector array comprised 384 solid state detectors, each 1.8 mm×28 mm, operating in the current mode. The x-ray tube was collimated to provide a fan beam of photons expanding to fill the field of view of the linear array in the transverse direction and a beam of width of 1 cm at the center of the scan field in the axial direction.

The x-ray CT data was acquired just prior to nuclear magnetic (NM) imaging studies on the same scanner and using the same imaging bed. A fixed linear translation was applied to the NM data, which represented the motion of the imaging be between the CT and NM studies. Application of this translation resulted in registered CT and NM images. X-ray CT and NM imaging was performed with two rats lying in serial fashion (nose to nose) on a low-density 3-foot diameter section of a tubular cardboard support and aligned with the axis of rotation of the NM/CT system. The low-density support minimized photon backscatter and attenuation effects. Medium energy collimators were used, which represented a compromise between image resolution and sensitivity. Following x-ray data acquisition, ¹³¹I SPECT data were acquired using a 128×128×64 acquisition matrix with a step-and-shoot approach (6° increments, 60 seconds per view) for a total scan time of 30 minutes. The distance between the source (subject) and detector was minimized at 5 cm. Both CT and NM studies were then reconstructed using filtered back projection. The x-ray data was converted to equivalent attenuation coefficients at 364 kilo electron volts (keV) and used for non-uniform attenuation compensation of the SPECT images. See Chang, 1983 and Webb et al., 1983. Due to the combination of a small-sized scattering medium and relative high-energy photons, scatter correction was not performed on the SPECT data. A post-reconstruction deconvolution filtering approach was used to approximately compensate for the degrading effects of geometric detector response, as described by Metz, 1969.

Animal subjects were first positioned for x-ray transmission scanning and up to 40 transverse image slices were obtained as the animals were indexed through the imaging field using a computer-controlled imaging table. During x-ray transmission scanning, the system was rotated at 2.6 revolutions per minute. Forty slices were acquired in approximately 9.0 minutes (13.8 seconds per slice). At the completion of the x-ray transmission scan, the computer-controlled imaging table was repositioned so that the axial field of view of the x-ray data was registered with the axial field of view of the dual-head scintillation camera. A 30-minute SPECT scan was then acquired using a step-and-shoot approach. X-ray transmission data was reconstructed into a 128×128×number of image slices (pixel size=3.1 mm) matrix to correspond to the array size of the reconstructed SPECT scan to facilitate attenuation compensation.

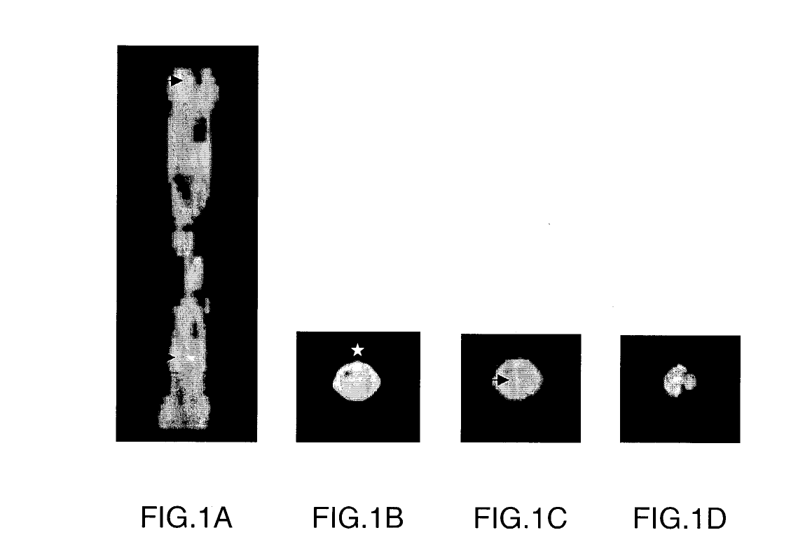

Rat subjects were imaged at 1 hour and at 24 hours after vector administration using the medium energy collimator CT/SPECT (HAWKEYE™ model available from GE Medical Systems, Waukesha, Wis., United States of America). CT and SPECT data were sequentially obtained while rats were anesthetized in the same position. After tail vein injection, radiohalogenated vector localized to the spleen (FIGS. 1A-1C). One hour after intratumoral injection, little activity was detected in the blood and the tumor was the predominant region showing radiohalogenated vector (FIG. 1D). At 24 hours after vector administration, radiohalogenated vector was not detected in the blood; however, thyroid uptake was observed.

Statistical analysis was used to describe differences among experimental groups. In general, a sample size of eight per group gave about 80% of power to detect a difference of 1.5-fold standard deviation. (Hallahan et al., 1995b; Seung et al., 1995). The statistical analyses were completed using the Statistical Analysis System (SAS) version 6.12 statistical analysis program (SAS Institute Inc., Cary, N.C., United States of America).

The statistical analysis focused on the use on non-invasive imaging technique and mathematical models to measure the pharmacokinetics (PK) and pharmacodynamics (PD) of the targeting technique. Pharmacokinetic parameters were presented in tabular and graphic form and included factors such as maximal plasma concentration, time of maximal concentration, and area under the plasma concentration time curve. Statistical analyses were performed using the General Linear Model method of the SAS software. If insignificant differences were indicated by the Analysis of Variance (ANOVA) analysis, the Waller-Duncan K-ration t-test was used for pairwise comparisons of mean pharmacokinetic parameter values.

For single time point data, a correlation between imaging results and PK or PD results was tested using the paired t-test of Wilcoxon Signed-Rank test for continuous parameters, or the McNemar's Chi-square test for categorical parameters.

For count and binary multiple time points data, a potential correlation between imaging results and pharmacokinetic or pharmacodynamic results was tested using the Generalized Estimating Equation (GEE) method statistical procedure for longitudinal data analysis with multiple observable vectors for the same subject (Liang & Zeger, 1986; Diggle et al., 1994).

For continuous multiple time points data, a potential correlation between groups was tested using the restricted/residual maximum likelihood (REML)-based repeated measure model (mixed model analysis) (Jennrich & Schluchter, 1986) with various covariance structure.

To optimize imaging of gene therapy vectors, various modifications are introduced, including but not limited to: (1) increasing septal thickness on the SPECT collimator; (2) reducing the distance of the collimator to the subject; (3) increasing the CT matrix to 256×256; (4) administering Lugol's iodine solution prior to vector administration; and combinations thereof.

For example, the effect of administering Lugol's iodine solution is tested as follows. 293 cells are infected with adenovirus encoding ExFlk.6His and are incubated with ¹³¹IUdR as described in Example 1. Excess ¹³¹IUdR is removed and cells are washed with phosphate-buffered saline. Radiohalogenated vector is harvested from the cultures and isolated using CsCl density gradients (Hallahan et al., 1995b; Lin et al., 1998b; Lin et al., 1998a). Lugol's iodine solution is added to drinking water during the 3 days prior to vector administration. Alternatively, Lugol's solution is administered by gavage. ¹³¹I-labeled adenovirus encoding ExFlk.6His is administered to tumor-bearing rats by tail vein injection (negative control) or intratumoral injection (positive control). Rats are imaged one day after vector administration.

The resolution of each SPECT image is compared to that obtained by phosphorimager plates, as described in Example 3, to assess whether modifying the collimator or the distance between the collimator and the subject improves the image resolution. Therapeutic protein expression is also measured by matrix-assisted laser desorption ionization/ time-of-flight mass spectrometry (MALDI-TOF) mass spectrometry to determine any effect of the modifications on detection of gene expression as described in Example 4. If a suboptimal correlation between SPECT images and imaging plates is observed, CT/SPECT resolution can be improved by changing the collimator and/or the CT matrix. Such improvements are made to achieve a correlation between SPECT images and direct measurements of ¹³¹I on imaging plates. Modifications that generate improved images are adopted.

### Example 3

Validation of CT/SPECT Imaging by Autoradiography

For micro-anatomical imaging and quantification, rats were sacrificed in accordance with the different protocols following injection of radiohalogenated vectors and antibodies. Immediately following sacrifice, the animals were frozen and a whole body cryomicrotome was used to cut the tissues into 50 mm thick sections (Yonekura et al., 1983). The tissue sections were placed in contact with the sensitive surface of the imaging medium. Sequential images were used to reconstruct the 3-dimensional (3-D) activity distributions throughout the sample tissues, especially in the tumor, and dose contributions between elements in the 3-D array were computed (Roberson et al., 1992; Humm et al., 1993; Koral et al., 1993; Roberson et al., 1994).

Fuji photo-stimulated luminescent imaging plates (IPs) were used for autoradiography (Fuji Medical Systems, Stamford, Conn., United States of America). The response (luminescent intensity) of the plates was linearly proportional to the activity (Amemiya et al., 1988; Mori et al., 1991). Less than one hour of exposure time was required to achieve readings equivalent to and optical densities (ODs) from 0 to 4.0 on film. The imaging plates were read at points spaced on a rectangular grid with spacing of either 50, 100, or 200 mm. The imaging plates were also sensitive to fluorescence from appropriately stained samples and the same scanner was used to read the plates having points spaced on a same rectangular grid. Because of the short exposure times for the plates, conventional film autoradiography was performed with the same tissue sections.

For conventional film autoradiography, the sections were placed in contact with the film (X-OMAT® XTL-2 film available from Eastman Kodak Co., Rochester, N.Y., United States of America). Both the mounted sections and the film were enclosed in a cassette with an image intensifier located directly behind and in contact with the film. For ¹³¹I, the measured signal enhancement from the intensifier degraded the image resolution by a small amount (final resolution to be approximately 70 mm). The film exposure times typically ranged from 10 to 15 hours in order to achieve relative ODs from 0.2 (fog) to 3.0.

Calibration data for both autoradiography media were obtained using radiolabeled gel or tissue-equivalent standards as described by Ito & Brill, 1990. Graded amounts of the radionuclide in question were mixed to homogeneity with a tissue paste or a 6% gelatin mixture. The specific activity of the mixture was established and serial dilutions are made. Each dilution was placed in a well with embedding material, then frozen. The frozen block containing the standard was cut into sections of the same thickness as the tissue sections and imaged in exactly the same way (Ito & Brill, 1990).

Section alignment was facilitated using radioactive markers embedded around the tumor. A coarse registration was achieved by calculating the optimum rotation matrix and translation vector that aligned the markers by a least-squares method (Arun et al., 1997). The registration was refined graphically by overlaying the image of each of the second and succeeding sections on the image of the preceding section, then rotating and translating it to achieve optimal alignment of the markers. For each voxel, the optical density was converted into an activity concentration.

Data obtained was expressed in units of the standard and was summed to indicate the total activity in the organ at the time of imaging, which was corrected to the time of sacrifice. By comparison with the gamma camera activity measurements (obtained as described in Example 2), the time activity curve was reconstructed and the tumor self-dose calculated (Roberson et al., 1992; Humm et al., 1993; Koral et al., 1993; Roberson et al., 1994).

### Example 4

Detection of Radiohalogen-Labeled Adenovirus Using MALDI-TOF Mass Spectrometry

To perform MALDI mass spectrometry, 12 mm thick tissue sections of a rat were prepared using a Leica CM 3000 cryostat (Leica Microsystems, Deerfield, Ill., United States of America) at −15° C. Sections were directly placed on a gold-coated stainless steel plate. The sections were transferred to a cold room set at 4° C., and 10 ml of matrix (sinapinic acid, 10 mg/ml in acetonitrile/0.05% trifluoro acetic acid 50:50) was deposited with a pipette as a line beside the tissue, and rapidly spread over the tissue using a small, flat plastic tool. After allowing crystallization for 45 minutes, the sections are dried for 2 hours in a desiccator. The application of matrix minimizes potential spreading of sample.

A MALDI mass spectrometer (VOYAGER ELITE™ model available from Applied Biosystems, Foster City, Calif., United States of America) was modified in both hardware and software as follows. The instrument had a delayed extraction option to achieve high sensitivity, a reflection to record high resolution mass spectra at low mass-to-charge ratio (m/z) values (<5000), and post-source decay capabilities to provide structure elucidation (e.g., for peptide sequencing). A nitrogen laser was used to irradiate the sample that had been mixed or had been coated with a crystalizable matrix material such as sinapinic acid (3,5-dimethoxy-4-hydroxycinnamic acid), CHCA (alpha-cyano-4-hydroxycinnamic acid) and DHBA (2,5-dihydroxybenzoic acid), matrices commonly used for labeling of peptides and proteins. The matrix was applied by directly pipetting a small volume of a saturated solution of matrix in aqueous ethanol or acetonitrile and allowing this preparation to dry. For high resolution imaging, the solution of matrix was applied directly on frozen tissue or by an electrospray process to achieve a uniform coating so that the spatial relationships of compounds were undisrupted. The sample holder was mounted in the target area on a movable stage that allowed repositioning of the area to be irradiated within fractions of a second. The original instrument was modified by the use of masks and lenses to narrow and shape the laser beam to a circular bean of diameter of about 25 μm at the target surface.

Imaging was accomplished from a raster of the surface of the sample by moving the sample stage. Each spot produced a mass spectrum that was the accumulation of about 100 laser shots at that spot. For high-resolution imaging, the laser-ablated spots were adjacent (on 25 μm steps). A survey was first obtained at 100 μm to 200 μm centers prior, and then a high-resolution image was obtained for a smaller area of interest. The mass spectrum of each laser spot usually contained hundreds of peaks, with accurate mass assignments (2 Dalton in 10,000 up to about 30,000 Daltons) for most of the peaks. Computer-generated images at a given molecular weight (a specific m/z value) were obtained by plotting the intensity value of the chosen molecular species in the ordered array or laser spots. Software, entitled the Mass Spectrometry Image Tool (MSIT), was written and implemented in order to facilitate and automate the imaging process. With this option, spot-to-spot cycles times were as rapid as one second, depending on the choice of user-determined parameters and sample quality, enabling a 3000 pixel image to be obtained in 50 minutes. This was not a limiting speed, and total acquisition times can be further improved by about a factor of 7 with hardware upgrades of commercially available high speed data transfer technology.

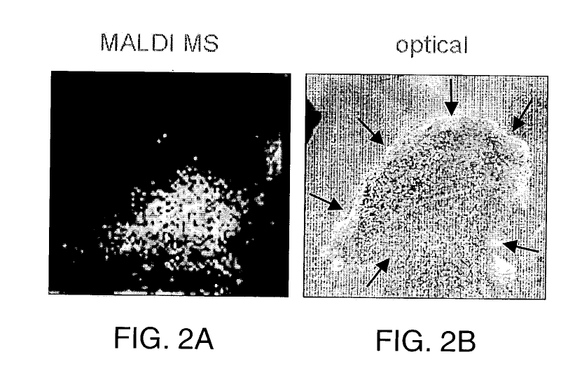

To determine the feasibility of using MALDI-TOF mass spectrometry to characterize vector biodistribution, C6 tumors from rat hind limb having received an injection of radiohalogenated vector were sectioned and analyzed by MALDI-TOF mass spectroscopy. The acquired images showed detection of therapeutic protein within the tumor (FIG. 2A).

### Example 5

Detection of a Recombinantly Expressed Heterologous Peptide Antigen

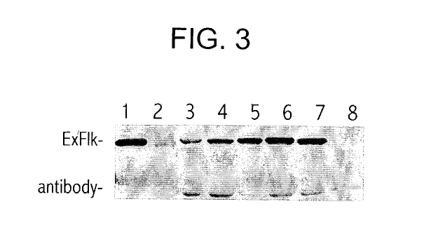

A recombinant soluble Flk-1 receptor, referred to herein as ExFlk.6His, was constructed by fusing the extracellular domain of murine flk-1 to a 6-histidine tag at the carboxyl terminus of Flk-1, as described by Lin et al., 1998. Adenovirus encoding ExFlk.6His was administered to a tumor-bearing rats subject by tail vein injection. On the following day, animals were sacrificed and ExFlk.6His expression was determined using a monoclonal ANTI-PENTA-HIS™ antibody (Qiagen, Inc., Valencia, Calif., United States of America) that specifically recognizes a peptide epitope comprising 5 contiguous histidine residues. ExFlk.6His polypeptides were immunoprecipitated using the PENTA-HIS™ antibody and were resolved by gel electrophoresis (FIG. 3). ExFlk.6His expression was detected in all tissues, and substantially no binding was observed in control animals.

The ExTek gene encodes the soluble portion of the TEK/Tie2 receptor, which binds the angiopoietin-1 ligand. ExTek was tagged with a peptide segment of streptavidin such that an anti-Strep-peptide antibody can be used to detect ExTek expression in transduced tissues. Anti-Strep-peptide antibody was labeled with ¹²⁵I using iodogen (available from NEN® Life Science Products, Inc., Boston, Mass., United States of America). The sequence of the strep peptide is IDARRASVGTSAWRHPQFGG (SEQ ID NO: 1).

Radiolabeled antibody (4 μCi of ¹²⁵I labeled antibody) or control IgG IV was injected into a mouse subject using tail vein injection 24 hours following administration of adenovirus encoding ExTek. On the next day, organs were isolated and well counts of ¹²⁵I were performed. Table 1 lists the counts per minute detected in each organ. Each value represents data collected from 5 Balb/c mice. The primary sites of ExTek detection were in the liver and the spleen. Detection of ExTek in the liver, spleen, and thyroid, was persistent to six days following antibody administration. Potential nonspecific binding of the antibody to bacteria in the intestines of the animal subjects was not observed. Counts observed in the thyroid were suspected to be uptake of free ¹²⁵I.

TABLE 1
¹²⁵I Antibody Distribution (CPM/control)

| | DAY 1 | | DAY 3 | | DAY 6 | |
|---|---|---|---|---|---|---|
| ORGAN | mean | SD | mean | SD | mean | SD |
| liver | 15.58 | 1.017 | 67.87 | 19.01 | 82.9 | 23.371 |
| spleen | 21.35 | 4.717 | 83.64 | 40.956 | 86.63 | 29.865 |
| lungs | 2.25 | 0.438 | 2.4 | 0.381 | 2.23 | 0.529 |
| heart | 1.96 | 0.186 | 2.01 | 0.323 | 2.12 | 0.224 |
| kidney | 5.35 | 0.39 | 8.16 | 0.96 | 9.95 | 1.5 |
| bladder | 1.97 | 0.351 | 1.65 | 0.328 | 2.16 | 1.937 |
| stomach | 9.43 | 1.798 | 5.86 | 0.934 | 5.27 | 1.194 |
| small intestine | 4.42 | 0.403 | 4.06 | 0.491 | 4.01 | 0.289 |
| large intestine | 7.93 | 1.714 | 5.64 | 1.676 | 7.82 | 1.878 |
| thyroid | 12.81 | 2.576 | 27.21 | 6.046 | 25.12 | 5.224 |
| muscle | 2.35 | 0.389 | 1.71 | 0.168 | 2.39 | 0.369 |
| blood | 1 | 0 | 1 | 0 | 1 | 0 |
| urine | 13.06 | 2.493 | 6.73 | 1.149 | 5.61 | 1.898 |
| tumor | 1.26 | 0.128 | 1.52 | 0.134 | 1.63 | 0.282 |
| bone | 3.14 | 0.194 | 3.37 | 0.473 | 4.16 | 1.326 |
| skin | 1.57 | 0.268 | 1.64 | 0.297 | 1.85 | 0.211 |
| brain | 3.5 | 0.72 | 2.24 | 0.25 | 2.8 | 0.696 |
| tail | 5.5 | 1.291 | 9.37 | 8.854 | 11.55 | 8.282 |
| total-TH | 4.69 | 0.4 | 8.86 | 1.929 | 10.89 | 1.508 |
| total | 8.73 | 1.668 | 19.44 | 1.723 | 21.31 | 4.041 |

CPM = counts per minute; SD = standard deviation

### Example 6

In Vivo Imaging of a Heterologous Peptide Antigen in Animal Models of Cancer

C6 and 3230AC tumors were implanted into subcutaneous tissue of Wistar and Fischer 344 rat hind limbs, and were grown to a volume of 1 cm. Lugol's iodine solution was administered to rat subjects to prevent thyroid uptake and reduce nonspecific ¹³¹I uptake. X-ray-guided delivery using fibrinogen-coated liposomes were used as a model to study resolution, sensitivity and specificity of gene therapy imaging. Therapeutic vectors encoded Exflk.6His and ExTek.Strep. Empty vectors (lacking a therapeutic gene insert) were employed as positive and negative controls.

Animals injected with Exflk.6His were administered ¹³¹I-labeled ANTI-PENTA-HIS™ antibody (Qiagen, Inc., Valencia, Calif., United States of America). Animals that were administered ExTek.Strep were subsequently administered ¹³¹I-labeled anti-Strep antibody. Antibodies were administered by tail vein injection. CT/SPECT images were collected as described in Example 2. Following imaging, whole animals were sectioned so that SPECT imaging can be compared to autoradiography (Example 3) and immunofluorescence histology. Eight (8) rats were used for each experimental group and statistical analysis is performed as described in Example 2.

To validate in vivo detection of a recombinantly expressed heterologous antigen, portions of organs and tumors were homogenized and ELISA was performed as described previously (Hallahan et al., 1995b; Seung et al., 1995; Staba et al., 1998). Tissue sections were fixed and prepared for immunofluorescence as previously described (Hallahan et al., 1995b; Advani et al., 1998; Lin et al., 1998a). Methods for immunofluorescent detection of ExFlk.6His and ExTek.Strep are described by (Hallahan et al., 1995b; Seung et al., 1995; Staba et al., 1998).

References

The references listed below as well as all references cited in the specification are incorporated herein by reference to the extent that they supplement, explain, provide a background for or teach methodology, techniques and/or compositions employed herein.

Abruzzese R V, Godin D, Burcin M, Mehta V, French M, Li Y, O'Malley B W & Nordstrom J L (1999) Ligand-dependent regulation of plasmid-based transgene expression in vivo. Hum Gene Ther 10:1499-1507.

Advani S J, Sibley G S, Song P Y, Hallahan D E, Kataoka Y, Roizman B & Weichselbaum R R (1998) Enhancement of replication of genetically engineered herpes simplex viruses by ionizing radiation: a new paradigm for destruction of therapeutically intractable tumors. Gene Ther 5:160-165.

Albini A, Marchisone C, Del Grosso F, Benelli R, Masiello L, Tacchetti C, Bono M, Ferrantini M, Rozera C, Truini M, Belardelli F, Santi L & Noonan D M (2000) Inhibition of angiogenesis and vascular tumor growth by interferon-producing cells: A gene therapy approach. Am J Pathol 156:1381-1393.

Amemiya Y, Satow Y, Matsushita T, J. Chikawa, Wakabayashi K & Miyahara J (1988) A Storage Phosphor Detector (Imaging Plate) and its Application to Diffraction Studies Using Synchrotron Radiation. In: Topics in Current Chemistry. Springer-Verlag, Heidelberg.

Andersson L, Blomberg L, Flegel M, Lepsa L, Nilsson B & Verlander M (2000) Large-scale synthesis of peptides. Biopolymers 55:227-250.

Arun K S, Huang T S & Blostein S D (1997) Least-Squares Fitting of Two 3-D Point Sets. PAMI:9698-9700.

Attal J, Theron M C & Houdebine L M (1999a) The optimal use of IRES (internal ribosome entry site) in expression vectors. Genet Anal 15:161-165.

Attal J, Theron M C, Puissant C & Houdebine L M (1999b) Effect of intercistronic length on internal ribosome entry site (IRES) efficiency in bicistronic mRNA. Gene Expr 8:299-309.

Bauminger S & Wilchek M (1980) The use of carbodiimides in the preparation of immunizing conjugates. Methods Enzymol70:151-159.

Betageri G, Jenkins S & Parsons D (1993) Liposome Drug Delivery Systems. Technomic Publishing, Lancaster.

Bodanszky M (1993) Principles of Peptide Synthesis, 2nd rev. ed. Springer-Verlag, Berlin; New York.

Brill A & Karellas (1991)—In: New trends in pharmacokinetics. p 147. Plenum Press, New York.

Burcin M M, Schiedner G, Kochanek S, Tsai S Y & O'Malley B W (1999) Adenovirus-mediated regulable target gene expression in vivo. Proc Natl Acad Sci USA 96:355-360.

Carpizo D & Iruela-Arispe M L (2000) Endogenous regulators of angiogenesis—emphasis on proteins with thrombospondin—type I motifs. Cancer Metastasis Rev 19:159-165.

Chang L T (1983) A method for attenuation correction in radionuclide computed tomography. IEEE Trans Nucl Sci NS-25:638-643.

Chappell S A, Edelman G M & Mauro V P (2000) A 9-nt segment of a cellular mRNA can function as an internal ribosome entry site (IRES) and when present in linked multiple copies greatly enhances IRES activity. Proc Natl Acad Sci USA 97:1536-1541.

Cheng P W (1996) Receptor ligand-facilitated gene transfer: enhancement of liposome-mediated gene transfer and expression by transferrin. Hum Gene Ther 7:275-282.

Cheng S, Craig W S, Mullen D, Tschopp J F, Dixon D & Pierschbacher M D (1994) Design and synthesis of novel cyclic RGD-containing peptides as highly potent and selective integrin alpha IIb beta 3 antagonists. J Med Chem 37:1-8.

Clapp C, Martial J A, Guzman R C, Rentier-Delure F & Weiner R I (1993) The 16-kilodalton N-terminal fragment of human prolactin is a potent inhibitor of angiogenesis. Endocrinology 133:1292-1299.

Coatney R W (2001) Ultrasound imaging: principles and applications in rodent research. Ilar J 42:233-247.

Cohen J I & Seidel K E (1993) Generation of varicella-zoster virus (VZV) and viral mutants from cosmid DNAs: VZV thymidylate synthetase is not essential for replication in vitro. Proc Natl Acad Sci USA 90:7376-7380.

Corringer P J, Weng J H, Ducos B, Durieux C, Boudeau P, Bohme A & Roques B P (1993) CCK-B agonist or antagonist activities of structurally hindered and peptidase-resistant Boc-CCK4 derivatives. J Med Chem 36:166-172.

Cruz P E, Goncalves D, Almeida J, Moreira J L & Carrondo M J (2000a) Modeling retrovirus production for gene therapy. 2. Integrated optimization of bioreaction and downstream processing. Biotechnol Prog 16:350-357.

Csermely P, Schnaider T, Soti C, Prohaszka Z & Nardai G (1998) The 90-Kda Molecular Chaperone Family: Structure, Function, and Clinical Applications. A Comprehensive Review. Pharmacol Ther79:129-168.

Cruz P E, Almeida J S, Murphy P N, Moreira J L & Carrondo M J (2000b) Modeling retrovirus production for gene therapy. 1. Determination Of optimal bioreaction mode and harvest strategy. Biotechnol Prog 16:213-221.

Cunningham C & Davison A J (1993) A cosmid-based system for constructing mutants of herpes simplex virus type 1. Virology 197:116-124.

Dameron K M, Volpert O V, Tainsky M A & Bouck N (1994) Control of angiogenesis in fibroblasts by p53 regulation of thrombospondin-1. Science 265:1582-1584.

Datz F L, Anderson C E, Ahluwalia R, Whiting J H, Gabor F V, Morton K A, Christian P E, Crebs K, Neptune M & Rauh D A (1994) The efficacy of indium-111-polyclonal IgG for the detection of infection and inflammation. J Nucl Med 35:74-83.

de Feyter R & Li P (2000) Technology evaluation: HIV ribozyme gene therapy, Gene Shears Pty Ltd. Curr Opin Mol Ther 2:332-335.

Delecluse H J & Hammerschmidt W (2000) The genetic approach to the Epstein-Barr virus: from basic virology to gene therapy. Mol Pathol 53:270-279.

Deuschle U, Meyer W K & Thiesen H J (1995) Tetracycline-reversible silencing of eukaryotic promoters. Mol Cell Biol 15:1907-1914.

Dias S, Thomas H & Balkwill F (1998) Multiple molecular and cellular changes associated with tumour stasis and regression during IL-12 therapy of a murine breast cancer model. Int J Cancer 75:151-157.

Diggle P, Liang K Y & Zeger S L (1994) Analysis of longitudinal data. Clarendon Press, Oxford.

Easton D P, Kaneko Y & Subjeck J R (2000) The HSP110 and GRP1 70 Stress Proteins: Newly Recognized Relatives of the HSP70s. Cell Stress Chaperones 5:276-290.

Ehsan A & Mann M J (2000) Antisense and gene therapy to prevent restenosis. Vasc Med 5:103-114.

Eijan A M, Davel L, Oisgold-Daga S & de Lustig E S (1991) Modulation of tumor-induced angiogenesis by proteins of extracellular matrix. Mol Biother 3:38-40.

European Patent No. 0 439 095

Fabbro D, Ruetz S, Bodis S, Pruschy M, Csermak K, Man A, Campochiaro P, Wood J, O'Reilly T & Meyer T (2000) PKC412—a protein kinase inhibitor with a broad therapeutic potential. Anticancer Drug Des 15:17-28.

Fields G B & Noble R L (1990) Solid phase peptide synthesis utilizing 9-fluorenylmethoxycarbonyl amino acids. Int J Pept Protein Res 35:161-214.

Fraser S E (1996) lontophoretic dye labeling of embryonic cells. Methods Cell Biol 51:147-160.

Freireich E J, Gehan E A, Rail D P, Schmidt L H & Skipper H E (1966) Quantitative comparison of toxicity of anticancer agents in mouse, rat, hamster, dog, monkey, and man. Cancer Chemother Rep 50:219-244.

Furler S, Paterna J C, Weibel M & Bueler H (2001) Recombinant AAV vectors containing the foot and mouth disease virus 2A sequence confer efficient bicistronic gene expression in cultured cells and rat substantia nigra neurons. Gene Ther 8:864-873.

Garbay-Jaureguiberry C, Ficheux D & Roques B P (1992) Solid phase synthesis of peptides containing the non-hydrolyzable analog of (O)phosphotyrosine, p(CH2PO3H2)Phe. Application to the synthesis of 344-357 sequences of the beta 2 adrenergic receptor. Int J Pept Protein Res 39:523-527.

Gerner E W, Hersh E M, Pennington M, Tsang T C, Harris D, Vasanwala F & Brailey J (2000) Heat-inducible vectors for use in gene therapy. Int J Hyperthermia 16:171-181.

Goldman C K, Kendall R L, Cabrera G, Soroceanu L, Heike Y, Gillespie G Y, Siegal G P, Mao X, Bett A J, Huckle W R, Thomas K A & Curiel D T (1998) Paracrine expression of a native soluble vascular endothelial growth factor receptor inhibits tumor growth, metastasis, and mortality rate. Proc Natl Acad Sci USA 95:8705-8800.

Goldman C K, Rogers B E, Douglas J T, Sosnowski B A, Ying W, Siegal G P, Baird A, Campain J A & Curiel D T (1997) Targeted gene delivery to Kaposi's sarcoma cells via the fibroblast growth factor receptor. Cancer Res 57:1447-1451.

Gossen M & Bujard H (1992) Tight Control of Gene Expression in Mammalian Cells by Tetracycline-Responsive Promoters. Proc Natl Acad Sci USA 89:5547-5551.

Gossen M & Bujard H (1993) Anhydrotetracycline, a Novel Effector for Tetracycline Controlled Gene Expression Systems in Eukaryotic Cells. Nucleic Acids Res 21:4411-4412.

Gossen M, Freundlieb S, Bender G, Muller G, Hillen W & Bujard H (1995) Transcriptional Activation by Tetracyclines in Mammalian Cells. Science 268:1766-1769.

Gregoriadis G, ed (1993) Liposome Technology. CRC Press, Boca Raton, Fla.

Griscelli F, Li H, Cheong C, Opolon P, Bennaceur-Griscelli A, Vassal G, Soria J, Soria C, Lu H, Perricaudet M & Yeh P (2000) Combined effects of radiotherapy and angiostatin gene therapy in glioma tumor model. Proc Natl Acad Sci USA 97:6698-6703.

Guha C, Chowdhury N R & Chowdhury J R (2000) Recombinant adenoassociated virus in cancer gene therapy. J Hepatol 32:1031-1034.

Habib N A, Hodgson H J, Lemoine N & Pignatelli M (1999) A phase I/II study of hepatic artery infusion with wtp53-CMV-Ad in metastatic malignant liver tumours. Hum Gene Ther 10:2019-2034.

Halbert C L, Alexander I E, Wolgamot G M & Miller A D (1995) Adeno-associated virus vectors transduce primary cells much less efficiently than immortalized cells. J Virol 69:1473-1479.

Hallahan D E, Geng L, Cmelak A J, Chakravarthy A B, Martin W, Scarfone C & Gonzalez A (2001) Targeting drug delivery to radiation-induced neoantigens in tumor microvasculature. J Control Release 74:183-191.

Hallahan D, Clark E T, Kuchibhotla J, Gewertz B L & Collins T (1995a) E-Selectin Gene Induction by Ionizing Radiation Is Independent of Cytokine Induction. Biochem Biophys Res Commun 217:784-795.

Hallahan D E, Mauceri H J, Seung L P, Dunphy E J, Wayne J D, Hanna N N, Toledano A, Hellman S, Kufe D W & Weichselbaum R R (1995b) Spatial and temporal control of gene therapy using ionizing radiation. Nat Med 1:786-791.

Harlow E & Lane D (1988) Antibodies: A Laboratory Manual. Cold Spring Harbor Laboratory Press, Cold Spring Harbor, N.Y.

Harries M, Phillipps N, Anderson R, Prentice G & Collins M (2000) Comparison of bicistronic retroviral vectors containing internal ribosome entry sites (IRES) using expression of human interleukin-12 (IL-12) as a readout. J Gene Med 2:243-249.

Hauck W & Stanners C P (1995) Transcriptional Regulation of the Carcinoembryonic Antigen Gene. Identification of Regulatory Elements and Multiple Nuclear Factors. J Biol Chem 270:3602-3610.

Havenga M J, Vogels R, Braakman E, Kroos N, Valerio D, Hagenbeek A & van Es H H (1998) Second gene expression in bicistronic constructs using short synthetic intercistrons and viral IRES sequences. Gene 222:319-327.

Heller R, Jaroszeski M, Atkin A, Moradpour D, Gilbert R, Wands J & Nicolau C (1996) In vivo gene electroinjection and expression in rat liver. FEBS Lett 389:225-228.

Hennecke M, Kwissa M, Metzger K, Oumard A, Kroger A, Schirmbeck R, Reimann J & Hauser H (2001) Composition and arrangement of genes define the strength of IRES-driven translation in bicistronic mRNAs. Nucleic Acids Res 29:3327-3334.

Heredia M, Santacana M & Valverde F (1991) A method using DiI to study the connectivity of cordical transplants. J Neurosci Methods 36:17-25.

Hitt M M & Graham F L (2000) Adenovirus vectors for human gene therapy. Adv Virus Res 55:479-505.

Hofmann G A, Dev S B, Nanda G S & Rabussay D (1999) Electroporation therapy of solid tumors. Crit Rev Ther Drug Carrier Syst 16:523-569.

Humm J L, Macklis R M, Bump K, Cobb L M & Chin L M (1993) Internal dosimetry using data derived from autoradiographs. J Nucl Med 34:1811-1817.

Hurley J H & Misra S (2000) Signaling and subcellular targeting by membrane-binding domains. Annu Rev Biophys Biomol Struct 29:49-79.

Ingber D, Fujita T, Kishimoto S, Sudo K, Kanamaru T, Brem H & Folkman J (1990) Synthetic analogues of fumagillin that inhibit angiogenesis and suppress tumour growth. Nature 348:555-557.

Ito T & Brill A B (1990) Validity of tissue paste standards for quantitative whole-body autoradiography using short-lived radionuclides. Int J Rad Appl Instrum [A] 41:661-667.

Janoff A, ed (1999) Liposomes: Rational Design. M. Dekker, New York.

Jennrich R I & Schluchter M D (1986) Unbalanced repeated-measures models with structured covariance matrices. Biometrics 42:805-820.

Jespersen T, Duch M, Carrasco M L, Warming S & Pedersen F S (1999) Expression of heterologous genes from an IRES translational cassette in replication competent murine leukemia virus vectors. Gene 239:227-235.

Johnson J E, Giorgione J & Newton A C (2000) The C1 and C2 domains of protein kinase C are independent membrane targeting modules, with specificity for phosphatidylserine conferred by the C1 domain. Biochemistry 39:11360-11369.

Joki T, Nakamura M & Ohno T (1995) Activation of the Radiosensitive Egr-1 Promoter Induces Expression of the Herpes Simplex Virus Thymidine Kinase Gene and Sensitivity of Human Glioma Cells to Ganciclovir. Hum Gene Ther 6:1507-1513.

Karas M & Heillenkamp F (1988) Laser lesorption ionization of proteins with molecular masses exceeding 10,000 daltons. Anal Chem 60:2299-2301.

Karas M, Bachmann D, Bahr U & Hillenkamp F (1987) Matrix-assisted ultraviolet-laser desorption of nonvolatile compounds. Int J Mass Spectom Ion Processes 78:53-68.

Kirk C J & Mule J J (2000) Gene-modified dendritic cells for use in tumor vaccines. Hum Gene Ther 11:797-806.

Kirpotin D, Park J W, Hong K, Zalipsky S, Li W L, Carter P, Benz C C & Papahadjopoulos D (1997) Sterically stabilized anti-HER2 immunoliposomes: design and targeting to human breast cancer cells in vitro. Biochemistry 36:66-75. Klump H, Schiedimeier B, Vogt B, Ryan M, Ostertag W & Baum C (2001) Retroviral vector-mediated expression of HoxB4 in hematopoietic cells using a novel coexpression strategy. Gene Ther 8:811-817.

Komaki S & Vos J M (2000) Epstein-Barr virus vectors for gene therapy. Adv Virus Res 55:453-462.

Koning G A, Morselt H W, Velinova M J, Donga J, Gorter A, Allen T M, Zalipsky S, Kamps J A & Scherphof G L (1999) Selective transfer of a lipophilic prodrug of 5-fluorodeoxyuridine from immunoliposomes to colon cancer cells. Biochim Biophys Acta 1420:153-167.

Koral K F, Kwok C S, Yang F E, Brown R S, Sisson J C & Wahl R L (1993) Autoradiography-based, three-dimensional calculation of dose rate for murine, human-tumor xenografts. Nucl Med Biol 20:901-909.

Kosfeld M D & Frazier W A (1993) Identification of a new cell adhesion motif in two homologous peptides from the COOH-terminal cell binding domain of human thrombospondin. J Biol Chem 268:8808-8814.

Krauzewicz N & Griffin B E (2000) Polyoma and papilloma virus vectors for cancer gene therapy. Adv Exp Med Biol 465:73-82.

Labat-Moleur F, Steffan A M, Brisson C, Perron H, Feugeas O, Furstenberger P, Oberling F, Brambilla E & Behr J P (1996) An electron microscopy study into the mechanism of gene transfer with lipopolyamines. Gene Ther 3:1010-1017.

Lasic D & Martin F, eds (1995) Stealth Liposomes. CRC Press, Boca Raton, Fla.

Latchman D S (2001) Gene delivery and gene therapy with herpes simplex virus-based vectors. Gene 264:1-9.

Lees W (2001) Ultrasound imaging in three and four dimensions. Semin Ultrasound CT MR 22:85-105.

Lemmon M A & Ferguson K M (2000) Signal-dependent membrane targeting by pleckstrin homology (PH) domains. Biochem J 350 Pt 1:1-18.

Lerondel S, Le Pape A, Sene C, Faure L, Bernard S, Diot P, Nicolis E, Mehtali M, Lusky M, Cabrini G & Pavirani A (2001) Radioisotopic imaging allows optimization of adenovirus lung deposition for cystic fibrosis gene therapy. Hum Gene Ther 12:1-11.

Leserman L D, Barbet J, Kourilsky F & Weinstein J N (1980) Targeting to cells of fluorescent liposomes covalently coupled with monoclonal antibody or protein A. Nature 288:602-604.

Lewin A S & Hauswirth W W (2001) Ribozyme gene therapy: applications for molecular medicine. Trends Mol Med 7:221-228.

Liang K & Zeger S L (1986) Longitudinal data analysis using generalized linear models. Biometrika 73:13-22.

Licha K, Riefke B, Ntziachristos V, Becker A, Chance B & Semmler W (2000) Hydrophilic cyanine dyes as contrast agents for near-infrared tumor imaging: synthesis, photophysical properties and spectroscopic in vivo characterization. Photochem Photobiol 72:392-398.

Lin P, Sankar S, Shan S, Dewhirst M W, Polverini P J, Quinn T Q & Peters K G (1998a) Inhibition of tumor growth by targeting tumor endothelium using a soluble vascular endothelial growth factor receptor. Cell Growth Differ 9:49-58.

Lin P. Buxton J A. Acheson A, Radziejewski C, Maisonpierre P C, Yancopoulos G D, Channon K M, Hale L P, Dewhirst M W, George S E & Peters K G (1998b) Antiangiogenic gene therapy targeting the endothelium-specific receptor tyrosine kinase Tie2. Proc Natl Acad Sci USA 95:8829-8834.

Lundstrom K (1999) Alphaviruses as tools in neurobiology and gene therapy. J Recept Signal Transduct Res 19:673-686.

Mackensen A, Lindemann A & Mertelsmann R (1997) Immunostimulatory cytokines in somatic cells and gene therapy of cancer. Cytokine Growth Factor Rev 8:119-128.

MacLaren D C, Gambhir S S, Satyamurthy N, Barrio J R, Sharfstein S, Toyokuni T, Wu L, Berk A J, Cherry S R, Phelps M E & Herschman H R (1999) Repetitive, non-invasive imaging of the dopamine D2 receptor as a reporter gene in living animals. Gene Ther 6:785-791.

Madio D P, van Gelderen P, DesPres D, Olson A W, de Zwart J A, Fawcett T W, Holbrook N J, Mandel M & Moonen C T (1998) On the feasibility of MRI-guided focused ultrasound for local induction of gene expression. J Magn Reson Imaging 8:101-104.

Maione T E, Gray G S, Petro J, Hunt A J, Donner A L, Bauer S I, Carson H F & Sharpe R J (1990) Inhibition of angiogenesis by recombinant human platelet factor-4 and related peptides. Science 247:77-79.

Manome Y, Abe M, Hagen M F, Fine H A & Kufe D W (1994) Enhancer sequences of the DF3 gene regulate expression of the herpes simplex virus thymidine kinase gene and confer sensitivity of human breast cancer cells to ganciclovir. Cancer Res 54:5408-5413.

Marin M, Noel D & Piechaczyk M (1997) Towards efficient cell targeting by recombinant retroviruses. Mol Med Today 3:396-403.

McOmie J F W (1973) Protective groups in organic chemistry. Plenum Press, London, N.Y.

Merrifield R B (1969) Solid-phase peptide synthesis. Adv Enzymol Relat Areas Mol Biol 32:221-296.

Metz C E (1969) A Mathematical Investigation of Radioisotope Scan Image Processing. Doctoral Dissertation, University of Pennsylvania.

Miklavcic D, Beravs K, Semrov D, Cemazar M, Demsar F & Sersa G (1998) The importance of electric field distribution for effective in vivo electroporation of tissues. Biophys J 74:2152-2158.

Mori S, Tanaka R & Minakawa T (1991) Effects of radiation on capillary endothelial cells derived from Mongolian gerbil brain. Neurosurgery 29:658-661; discussion 662.

Morishita R, Aoki M & Kaneda Y (2000) Oligonucleotide-based gene therapy for cardiovascular disease: are oligonucleotide therapeutics novel cardiovascular drugs? Curr Drug Targets 1:15-23.

Nabel G (1997) Vectors for Gene Therapy. In: Current Protocols in Human Genetics. John Wiley & Sons, New York.

Nam S M, Kim H S, Ahn W S & Park Y S (1999) Sterically stabilized anti-G(M3), anti-Le(x) immunoliposomes: targeting to B16BL6, HRT-18 cancer cells. Oncol Res 11:9-16.

Narvaiza I, Mazzolini G, Barajas M, Duarte M, Zaratiegui M, Qian C, Melero I & Prieto J (2000) Intratumoral coinjection of two adenoviruses, one encoding the chemokine IFN-gamma-inducible protein-10 and another encoding IL-12, results in marked antitumoral synergy. J Immunol 164:3112-3122.

Neri D, Carnemolla B, Nissim A, Leprini A, Querze G, Balza E, Pini A, Tarli L, Halin C, Neri P, Zardi L & Winter G (1997) Targeting by affinity-matured recombinant antibody fragments of an angiogenesis associated fibronectin isoform. Nat Biotechnol 15:1271-1275.

Neumann E, Schaefer-Ridder M, Wang Y & Hofschneider P H (1982) Gene transfer into mouse lyoma cells by electroporation in high electric fields. Embo J 1:841-845.

No D, Yao T P & Evans R M (1996) Ecdysone-inducible gene expression in mammalian cells and transgenic mice. Proc Natl Acad Sci USA 93:3346-3351.

Nomura T & Hasegawa H (2000) Chemokines and anti-cancer immunotherapy: anti-tumor effect of EBI1-ligand chemokine (ELC) and secondary lymphoid tissue chemokine (SLC). Anticancer Res 20:4073-4080.

Norris J S, Hoel B, Voeks D, Maggouta F, Dahm M, Pan W & Clawson G (2000) Design and testing of ribozymes for cancer gene therapy. Adv Exp Med Biol 465:293-301.

O'Byrne K J, Dalgleish A G, Browning M J, Steward W P & Harris A L (2000) The relationship between angiogenesis and the immune response in carcinogenesis and the progression of malignant disease. Eur J Cancer 36:151-169.

Ohtsuka K & Hata M (2000) Molecular Chaperone Function of Mammalian HSP70 and HSP40—a Review. Int J Hyperthermia 16:231-245.

O'Reilly M S, Holmgren L, Shing Y, Chen C, Rosenthal R A, Moses M, Lane W S, Cao Y, Sage E H & Folkman J (1994) Angiostatin: a novel angiogenesis inhibitor that mediates the suppression of metastases by a Lewis lung carcinoma. Cell 79:315-328.

O'Reilly M S, Boehm T, Shing Y, Fukai N, Vasios G, Lane W S, Flynn E, Birkhead J R, Olsen B R & Folkman J (1997) Endostatin: an endogenous inhibitor of angiogenesis and tumor growth. Cell 88:277-285.

Palese P, Zheng H, Engelhardt O G, Pleschka S & Garcia-Sastre A (1996) Negative-strand RNA viruses: genetic engineering and applications. Proc Natl Acad Sci USA 93:11354-11358.

Park J W, Hong K, Kirpotin D B, Papahadjopoulos D & Benz C C (1997) Immunoliposomes for cancer treatment. Adv Pharmacol 40:399-435.

Pasqualini R, Koivunen E & Ruoslahti E (1997) Alpha v integrins as receptors for tumor targeting by circulating ligands. Nat Biotechnol 15:542-546.

Pavone V, Di Blasio B, Lombardi A, Maglio O, Isernia C, Pedone C, Benedetti E, Altmann E & Mutter M (1993) Non coded C alpha, alpha-disubstituted amino acids. X-ray diffraction analysis of a dipeptide containing (S)-alpha-methylserine. Int J Pept Protein Res 41:15-20.

Phillips M I, Galli S M & Mehta J L (2000) The potential role of antisense oligodeoxynucleotide therapy for cardiovascular disease. Drugs 60:239-248.

Pomper M G & Port J D (2000) New techniques in MR imaging of brain tumors. Magn Reson Imaging Clin N Am 8:691-713.

Ragnarson B, Bengtsson L & Haegerstrand A (1992) Labeling with fluorescent carbocyanine dyes of cultured endothelial and smooth muscle cells by growth in dye-containing medium. Histochemistry 97:329-333.

Richards C A, Austin E A & Huber B E (1995) Transcriptional Regulatory Sequences of Carcinoembryonic Antigen: Identification and Use with Cytosine Deaminase for Tumor-Specific Gene Therapy. Hum Gene Ther 6:881-893.

Rigden J E, Ely J A, Macpherson J L, Gerlach W L, Sun L Q & Symonds G P (2000) The use of ribozyme gene therapy for the inhibition of HIV replication and its pathogenic sequelae. Curr Issues Mol Biol 2:61-69.

Roberson P L, Buchsbaum D J, Heidorn D B & Ten Haken R K (1992) Three-dimensional tumor dosimetry for radioimmunotherapy using serial autoradiography. Int J Radiat Oncol Biol Phys 24:329-334.

Roberson P L, Heidorn D B, Kessler M L, Ten Haken R K & Buchsbaum D J (1994) Three-dimensional reconstruction of monoclonal antibody uptake in tumor and calculation of beta dose-rate nonuniformity. Cancer 73:912-918.

Rossi J J (2000) Ribozyme therapy for HIV infection. Adv Drug Deliv Rev 44:71-78.

Rovaris M & Filippi M (2000) The role of magnetic resonance in the assessment of multiple sclerosis. J Neurol Sci 172 Suppl 1:S3-S12.

Sakamoto N, Iwahana M, Tanaka N G & Osada Y (1991) Inhibition of angiogenesis and tumor growth by a synthetic laminin peptide, CDPGYIGSR-NH2. Cancer Res 51:903-906.

Saltzman W M & Fung L K (1997) Polymeric implants for cancer chemotherapy. Adv Drug Deliv Rev 26:209-230.

Sandig V, Hofmann C, Steinert S, Jennings G, Schlag P & Strauss M (1996) Gene transfer into hepatocytes and human liver tissue by baculovirus vectors. Hum Gene Ther 7:1937-1945.

Sarkis C, Serguera C, Petres S, Buchet D, Ridet J L, Edelman L & Mallet J (2000) Efficient transduction of neural cells in vitro and in vivo by a baculovirus-derived vector. Proc Natl Acad Sci USA 97:14638-14643.

Schellingerhout D, Bogdanov A, Jr., Marecos E, Spear M, Breakefield X & Weissleder R (1998) Mapping the in vivo distribution of herpes simplex virions. Hum Gene Ther 9:1543-1549.

Schneider C H & Eberle A N (1993) Peptides, 1992: proceedings of the Twenty-Second European Peptide Symposium, Sep. 13-19, 1992 Interlaken, Switzerland. Escom, Leiden.

Schröder E & Lübke K (1965) The peptides. Academic Press, New York.

Sersa G, Stabuc B, Cemazar M, Miklavcic D & Rudolf Z (2000) Electrochemotherapy with cisplatin: clinical experience in malignant melanoma patients. Clin Cancer Res 6:863-867.

Seung L P, Mauceri H J, Beckett M A, Hallahan D E, Hellman S & Weichselbaum R R (1995) Genetic radiotherapy overcomes tumor resistance to cytotoxic agents. Cancer Res 55:5561-5565.

Shaughnessy E, Lu D, Chatterjee S & Wong K K (1996) Parvoviral vectors for the gene therapy of cancer. Semin Oncol 23:159-171.

Shen T, Weissleder R, Papisov M, Bogdanov A, Jr. & Brady T J (1993) Monocrystalline iron oxide nanocompounds (MION): physicochemical properties. Magn Reson Med 29:599-604.

Shimizu Y, Inoue A, Tomari Y, Suzuki T, Yokogawa T, Nishikawa K & Ueda T (2001) Cell-free translation reconstituted with purified components. Nat Biotechnol 19:751-755.

Shippy R, Lockner R, Farnsworth M & Hampel A (1999) The hairpin ribozyme. Discovery, mechanism, and development for gene therapy. Mol Biotechnol 12:117-129.

Silman N J & Fooks A R (2000) Biophysical targeting of adenovirus vectors for gene therapy. Curr Opin Mol Ther 2:524-531.

Simonova M, Weissleder R, Sergeyev N, Vilissova N & Bogdanov A, Jr. (1999) Targeting of green fluorescent protein expression to the cell surface. Biochem Biophys Res Commun 262:638-642.

Smith R C & Walsh K (2000) Gene therapy for restenosis. Curr Cardiol Rep 2:13-23.

Smith-Arica J R & Bartlett J S (2001) Gene therapy: recombinant adeno-associated virus vectors. Curr Cardiol Rep 3:43-49.

Srivastava A (1994) Parvovirus-based vectors for human gene therapy. Blood Cells 20:531-536.

Staba M J, Wickham T J, Kovesdi I & Hallahan D E (2000) Modifications of the fiber in adenovirus vectors increase tropism for malignant glioma models. Cancer Gene Ther 7:13-19.

Staba M J, Mauceri H J, Kufe D W, Hallahan D E & Weichselbaum R R (1998) Adenoviral TNF-alpha gene therapy and radiation damage tumor vasculature in a human malignant glioma xenograft. Gene Ther 5:293-300.

Stewart J M & Young J D (1969) Solid phase peptide synthesis. Freeman, San Francisco.

Takayama K, Ueno H, Nakanishi Y, Sakamoto T, Inoue K, Shimizu K, Oohashi H & Hara N (2000) Suppression of tumor angiogenesis and growth by gene transfer of a soluble form of vascular endothelial growth factor receptor into a remote organ. Cancer Res 60:2169-2177.

Tal J (2000) Adeno-associated virus-based vectors in gene therapy. J Biomed Sci 7:279-291.

Tolsma S S, Volpert O V, Good D J, Frazier W A, Polverini P J & Bouck N (1993) Peptides derived from two separate domains of the matrix protein thrombospondin-1 have anti-angiogenic activity. J Cell Biol 122:497-511.

Tung C H, Zhu T, Lackland H & Stein S (1992) An acridine amino acid derivative for use in Fmoc peptide synthesis. Pept Res 5:115-118.

Urge L, Otvos L, Jr., Lang E, Wroblewski K, Laczko I & Hollosi M (1992) Fmoc-protected, glycosylated asparagines potentially useful as reagents in the solid-phase synthesis of N-glycopeptides. Carbohydr Res 235:83-93.

U.S. Pat. No. 4,196,265

U.S. Pat. No. 4,235,871

U.S. Pat. No. 4,244,946

U.S. Pat. No. 4,551,482

U.S. Pat. No. 4,652,440

U.S. Pat. No. 4,671,958

U.S. Pat. No. 4,851,520

U.S. Pat. No. 4,946,778

U.S. Pat. No. 5,011,634

U.S. Pat. No. 5,017,359

U.S. Pat. No. 5,047,227

U.S. Pat. No. 5,080,883

U.S. Pat. No. 5,088,499

U.S. Pat. No. 5,091,513

U.S. Pat. No. 5,132,405

U.S. Pat. No. 5,147,631

U.S. Pat. No. 5,234,933

U.S. Pat. No. 5,260,203

U.S. Pat. No. 5,326,902

U.S. Pat. No. 5,328,679

U.S. Pat. No. 5,490,840

U.S. Pat. No. 5,499,971

U.S. Pat. No. 5,510,103

U.S. Pat. No. 5,574,172

U.S. Pat. No. 5,578,629

U.S. Pat. No. 5,597,531

U.S. Pat. No. 5,651,991

U.S. Pat. No. 5.677.427

U.S. Pat. No. 5,688,931

U.S. Pat. No. 5,703,056

U.S. Pat. No. 5,707,605

U.S. Pat. No. 5,714,166

U.S. Pat. No. 5,736,349

U.S. Pat. No. 5,738,837

U.S. Pat. No. 5,762,909

U.S. Pat. No. 5,762,918

U.S. Pat. No. 5,786,387

U.S. Pat. No. 5,811,392

U.S. Pat. No. 5,811,512

U.S. Pat. No. 5,811,515

U.S. Pat. No. 5,814,335

U.S. Pat. No. 5,817,757

U.S. Pat. No. 5,817,879

U.S. Pat. No. 5,851,818

U.S. Pat. No. 5,854,027

U.S. Pat. No. 5,855,900

U.S. Pat. No. 5,858,410

U.S. Pat. No. 5,865,754

U.S. Pat. No. 5,892,019

U.S. Pat. No. 5,922,356

U.S. Pat. No. 5,922,545

U.S. Pat. No. 5,928,627

U.S. Pat. No. 5,928,944

U.S. Pat. No. 5,948,767

U.S. Pat. No. 5,962,667

U.S. Pat. No. 5,965,106

U.S. Pat. No. 5,985,279

U.S. Pat. No. 5,994,392

U.S. Pat. No. 6,001,088

U.S. Pat. No. 6,015,561

U.S. Pat. No. 6,015,881

U.S. Pat. No. 6,024,938

U.S. Pat. No. 6,031,071

U.S. Pat. No. 6,041,253

U.S. Pat. No. 6,054,561

U.S. Pat. No. 6,056,938

U.S. Pat. No. 6,068,829

U.S. Pat. No. 6,071,890

U.S. Pat. No. 6,080,384

U.S. Pat. No. 6,083,486

U.S. Pat. No. 6,106,866

U.S. Pat. No. 6,127,339

U.S. Pat. No. 6,132,766

U.S. Pat. No. 6,136,295

U.S. Pat. No. 6,159,443

U.S. Pat. No. 6,165,440

U.S. Pat. No. 6,180,082

U.S. Pat. No. 6,180,084

U.S. Pat. No. 6,197,333

U.S. Pat. No. 6,200,598

U.S. Pat. No. 6,210,707

U.S. Pat. No. 6,217,886

U.S. Pat. No. 6,221,018

U.S. Pat. No. 6,231,834

U.S. Pat. No. 6,232,287

U.S. Pat. No. 6,234,990

U.S. Pat. No. 6,245,318

U.S. Pat. No. 6,246,901

U.S. Pat. No. 6,254,854

U.S. Pat. No. 6,254,852

U.S. Pat. No. 6,261,831

Vekris A, Maurange C, Moonen C, Mazurier F, De Verneuil H, Canioni P & Voisin P (2000) Control of transgene expression using local hyperthermia in combination with a heat-sensitive promoter. J Gene Med 2:89-96.

Vicat J M, Boisseau S, Jourdes P, Laine M, Wion D, Bouali-Benazzouz R, Benabid A L & Berger F (2000) Muscle transfection by electroporation with high-voltage and short- pulse currents provides high-level and long-lasting gene expression. Hum Gene Ther 11:909-916.

Vinogradov S A, Lo L W, Jenkins W T, Evans S M, Koch C & Wilson D F (1996) Noninvasive imaging of the distribution in oxygen in tissue in vivo using near-infrared phosphors. Biophys J 70:1609-1617.

Voest E E, Kenyon B M, O'Reilly M S, Truitt G, D'Amato R J & Folkman J (1995) Inhibition of angiogenesis in vivo by interleukin 12. J Natl Cancer Inst 87:581-586.

Wahlfors J J, Zullo S A, Loimas S, Nelson D M & Morgan R A (2000) Evaluation of recombinant alphaviruses as vectors in gene therapy. Gene Ther 7:472-480.

Walther W & Stein U (1999) Therapeutic genes for cancer gene therapy. Mol Biotechnol 13:21-28.

Webb S, Flower M A, Ott R J & Leach M O (1983) A comparison of attenuation correction methods for quantitative single photon emission computed tomography. Phys Med Biol 28:1045-1056.

Weichselbaum R R, Hallahan D, Fuks Z & Kufe D (1994) Radiation induction of immediate early genes: effectors of the radiation-stress response. Int J Radiat Oncol Biol Phys 30:229-234.

Weichselbaum R R, Hallahan D E, Sukhatme V, Dritschilo A, Sherman M L & Kufe D W (1991) Biological consequences of gene regulation after ionizing radiation exposure. J Natl Cancer Inst 83:480-484.

Weissleder R, Bogdanov A & Papisov M (1992) Drug targeting in magnetic resonance imaging. Magn Reson Q 8:55-63.

Weissleder R, Tung C H, Mahmood U & Bogdanov A, Jr. (1999) In vivo imaging of tumors with protease-activated near-infared fluorescent probes. Nat Biotechnol 17:375-378.

Wickham T J, Carrion M E & Kovesdi I (1995) Targeting of adenovirus penton base to new receptors through replacement of its RGD motif with other receptor-specific peptide motifs. Gene Ther 2:750-756.

WO 00/37502

WO 01/09611

WO 93/25521

WO 98/10795

WO 99/61071

Woltering E A, Barrie R, O'Dorisio T M, Arce D, Ure T, Cramer A, Holmes D, Robertson J & Fassler J (1991) Somatostatin analogues inhibit angiogenesis in the chick chorioallantoic membrane. J Surg Res 50:245-251.

Yang N S (1992) Gene transfer into mammalian somatic cells in vivo. Crit Rev Biotechnol 12:335-356.

Yeung S N & Tufaro F (2000) Replicating herpes simplex virus vectors for cancer gene therapy. Expert Opin Pharmacother 1:623-631.

Yonekura Y, Brill A B, Som P, Bennett G W & Fand I (1983) Quantitative autoradiography with radiopharmaceuticals, Part 1: Digital film-analysis system by videodensitometry: concise communication. J Nucl Med 24:231-237.

Yoo T M, Chang H K, Choi C W, Webber K O, Le N, Kim I S, Eckelman W C, Pastan I, Carrasquillo J A & Paik C H (1997) Technetium-99m labeling and biodistribution of anti-TAC disulfide- stabilized Fv fragment. J Nucl Med 38:294-300.

Zwiebel J A, Nagler C, Smiley J K & Cheson B D (1998) Clinical trails referral resource. Clinical trials of adenovirus-p53 gene therapy. Oncology (Huntingt) 12:1172, 1175.

It will be understood that various details of the invention can be changed without departing from the scope of the invention. Furthermore, the foregoing description is for the purpose of illustration only, and not for the purpose of limitation—the invention being defined by the claims appended hereto.

What is claimed is:

1. A method for preparing a halogen-labeled gene therapy construct, the method comprising:
(a) introducing a gene therapy construct into helper cells, wherein the gene therapy construct comprises one or more nucleic acids; and
(b) providing a halogen-labeled nucleotide to the helper cells, whereby a halogen-labeled gene therapy construct is prepared.

2. The method of claim 1, wherein the gene therapy construct comprises a viral vector, a plasmid, a liposome, or combinations thereof.

3. The method of claim 2, wherein the viral vector comprises an adenoviral vector.

4. The method of claim 1, wherein the one or more nucleic acids further comprises a nucleotide sequence encoding a therapeutic gene product.

5. The method of claim 1, wherein the halogen comprises a radiohalogen.

6. The method of claim 5, wherein the radiohalogen comprises ¹⁸F, ¹²³I, ¹²⁵I, or ¹³¹I.

7. The method of claim 1, wherein the halogen-labeled nucleotide comprises a pyrimidine nucleotide.

8. The method of claim 7, wherein the pyrimidine nucleotide comprises 2′-deoxyuridine.

9. The method of claim 1, further comprising isolating the halogen-labeled gene therapy construct from the helper cells.

10. A halogen-labeled gene therapy construct produced by the method of claim 1.

11. A halogen-labeled gene therapy construct comprising:
(a) a vector; and
(b) one or more nucleic acids, wherein one or more of the nucleic acids comprises a halogen-labeled nucleotide, wherein the nucleic acids are free of triplex structures, and wherein the halogen-labeled gene therapy construct can be detected in vivo.

12. The halogen-labeled gene therapy construct of claim 11, wherein the vector is selected from the group consisting of a viral vector, a plasmid, a liposome, and combinations thereof.

13. The halogen-labeled gene therapy construct of claim 12, wherein the viral vector comprises an adenoviral vector.

14. The halogen-labeled gene therapy construct of claim 11, wherein the one or more nucleic acids further comprises a nucleotide sequence encoding a therapeutic gene product.

15. The halogen-labeled gene therapy construct of claim 11, wherein the halogen comprises a radiohalogen.

16. The halogen-labeled gene therapy construct of claim 15, wherein the radiohalogen comprises ¹⁸F, ¹²³I, ¹²⁵I, or ¹³¹I.

17. The halogen-labeled gene therapy construct of claim 11, wherein the halogen-labeled nucleotide comprises a pyrimidine nucleotide.

18. The halogen-labeled gene therapy construct of claim 17, wherein the pyrimidine nucleotide comprises 2′-deoxyuridine.

19. A method for non-invasive detection in a subject of a halogen-labeled gene therapy construct, the method comprising:
(a) administering to a subject an effective dose of a halogen-labeled gene therapy construct, wherein the construct comprises a vector and one or more nucleic acids, and wherein one or more of the nucleic acids comprises a halogen-labeled nucleotide; and
(b) detecting the halogen-labeled nucleotide, wherein the detecting comprises a non-invasive detection technique.

20. The method of claim 19, wherein the subject is a warm-blooded vertebrate.

21. The method of claim 20, wherein the warm-blooded vertebrate is a human.

22. The method of claim 19, wherein the effective dose comprises a detectable amount of a halogen-labeled gene therapy construct, wherein the detectable amount is detected in a subject non-invasively.

23. The method of claim 19, wherein the vector comprises a viral vector, a plasmid, a liposome, or combinations thereof.

24. The method of claim 23, wherein the viral vector comprises an adenoviral vector.

25. The method of claim 19, wherein the one or more nucleic acids comprising a halogen-labeled nucleotide label are free of triplex structures.

26. The method of claim 19, wherein the one or more nucleic acids further comprises a nucleotide sequence encoding a therapeutic gene product.

27. The method of claim 19, wherein the halogen comprises a radiohalogen.

28. The method of claim 27, wherein the radiohalogen comprises ¹⁸F, ¹²³I, ¹²⁵I, or ¹³¹I.

29. The method of claim 19, wherein the halogen-labeled nucleotide comprises a pyrimidine nucleotide.

30. The method of claim 29, wherein the pyrimidine nucleotide comprises 2′-deoxyuridine.

31. The method of claim 19, wherein the detecting comprises detecting the halogen-labeled nucleotide using positron emission tomography, single photon emission computed tomography, gamma camera imaging, rectilinear scanning, or combinations thereof.

32. A method for detecting a drug in a subject, the method comprising:
(a) administering to a subject an effective dose of a drug, wherein the drug comprises a heterologous antigen;
(b) administering to the subject an antibody that binds the heterologous antigen, wherein the antibody comprises a label that can be detected in vivo; and
(c) detecting the label in vivo, whereby the drug is detected in the subject.

33. The method of claim 32, wherein the subject is a warm-blooded vertebrate.

34. The method of claim 33, wherein the warm-blooded vertebrate is a human.

35. The method of claim 32, wherein the effective dose comprises an amount of the drug that can be detected in vivo.

36. The method of claim 32, wherein the drug comprises a gene therapy construct, a small molecule, a protein, a peptide, a nucleic acid, a lipid, or combinations thereof.

37. The method of claim 36, wherein the gene therapy construct comprises a viral vector, a plasmid, a liposome, or combinations thereof.

38. The method of claim 37, wherein the viral vector comprises an adenoviral vector.

39. The method of claim 36, wherein the gene therapy construct comprises a nucleotide sequence encoding the heterologous antigen.

40. The method of claim 39, wherein the gene therapy construct further comprises a nucleotide sequence encoding a therapeutic gene product.

41. The method of claim 40, wherein the gene therapy construct further comprises a nucleotide sequence encoding a therapeutic gene product, and wherein the nucleotide sequence encoding a therapeutic gene product is operatively linked to the nucleotide sequence encoding the heterologous antigen.

42. The method of claim 32, wherein the heterologous antigen comprises a streptavidin peptide.

43. The method of claim 42, wherein the streptavidin peptide comprises an amino acid sequence of SEQ ID NO: 1.

44. The method of claim 32, wherein the heterologous antigen comprises a polyhistidine peptide.

45. The method of claim 44, wherein the polyhistidine peptide comprises an amino acid sequence of SEQ ID NO: 2.

46. The method of claim 32, wherein the label comprises a label that can be detected using magnetic resonance imaging, scintigraphic imaging, ultrasound, fluorescence, or combinations thereof.

47. The method of claim 46, wherein the label that can be detected using scintigraphic imaging comprises a radionuclide label.

48. The method of claim 47, wherein the radionuclide label comprises ¹⁸F, ¹²³I, ¹²⁵I, or ¹³¹I.

49. The method of claim 32, wherein the detecting comprises detecting the radionuclide label using positron emission tomography, single photon emission computed tomography, gamma camera imaging, rectilinear scanning, or combinations thereof.
